(19) United States
(12) Patent Application Publication
(10) Pub. No.: US 2025/0268523 A1
(43) Pub. Date: Aug. 28, 2025

(54) A SYSTEM CONFIGURED FOR CHRONIC ILLNESS MONITORING USING INFORMATION FROM MULTIPLE DEVICES

(71) Applicant: Medtronic, Inc., Minneapolis, MN (US)

(72) Inventors: Shantanu Sarkar, Roseville, MN (US); Todd J. Sheldon, North Oaks, MN (US); Wade M. Demmer, Coon Rapids, MN (US); Yong K. Cho, Excelsior, MN (US)

(21) Appl. No.: 18/858,668

(22) PCT Filed: Apr. 5, 2023
(86) PCT No.: PCT/IB2023/053479

§ 371 Date: Oct. 21, 2024

Related U.S. Application Data
(60) Provisional application No. 63/363,441, filed on Apr. 22, 2022.

(51) Int. Cl. A61B 5/00; A61B 5/0538; A61N 1/362; A61N 1/365; A61N 1/37; G16H 10/60; G16H 50/30

(52) U.S. Cl. CPC A61B 5/4836; A61B 5/0538; A61B 5/686; A61B 5/7264; A61B 5/7275; A61N 1/3627; A61N 1/36521; A61N 1/3702; G16H 10/60; G16H 50/30; A61B 2560/0468

(57) ABSTRACT

An example system includes a first implantable medical device (IMD), a second IMD and processing circuitry. The first IMD comprising one or more first sensors and configured to receive one or more first signals from the first sensors to determine one or more interstitial fluid index values based at least in part on the received first signals. The second IMD comprising one or more second sensors and configured to receive one or more second signals from the second sensors to determine one or more intravascular fluid index values based at least in part on the received second signals. The processing circuitry configured to detect a health condition status of the patient based at least in part on the interstitial fluid index values and the intravascular fluid index values.

This application claims the benefit of U.S. Provisional Patent Application Ser. No. 63/363,441, filed Apr. 22, 2022, the entire content of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, fluid impedance signals, and blood glucose or other blood constituent signals. In general, using these signals, such devices facilitate monitoring and evaluating patient health over a number of months or years, outside of a clinic setting. In some cases, some medical devices have been used or proposed to monitor heart failure (HF) or to detect HF events, such as arrhythmia or sudden cardiac arrest (SCA).

HF is the most common cardiovascular disease that causes significant economic burden, morbidity, and mortality. In the United States alone, roughly 5 million people have HF, accounting for a significant number of hospitalizations. HF may result in cardiac chamber dilation, increased pulmonary blood volume, and fluid retention. Acute decompensated HF is a manifestation of worsening HF or broadly chronic illness symptoms that requires HF admission to relieve patients of congestion and shortness of breath symptoms. Generally, the first indication that a physician has of worsening HF in a patient is not until it becomes a physical manifestation with swelling or breathing difficulties so overwhelming as to be noticed by the patient who then proceeds to be examined by a physician. This is undesirable since hospitalization at such a time would likely be required for a cardiac HF patient to remove excess fluid and relieve symptoms.

### SUMMARY

In general, the disclosure describes techniques for monitoring a chronic illness, such as HF or chronic obstructive pulmonary disease (COPD) by monitoring sensed patient data. More particularly, the disclosure describes techniques for obtaining the sensed patient data from a plurality of devices that provide orthogonal information, and detecting a health condition status of the patient, such as HF, worsening HF, risk of sudden cardiac death (SCD), COPD, renal failure, hypertension, sleep apnea, anemia, sepsis, or bleeding of the gut, based in part on the sensed patient data. The techniques may further include application of a machine learning model to the sensed patient data in order to improve the efficiency and effectiveness of the detection of a health condition status.

In some examples, a system having a first implantable medical device (IMD) to determine one or more interstitial fluid index values and second IMD to determine one or more intravascular fluid index values to detect a health condition status of a patient helps to detect serious health conditions such as HF, worsening HF, increased risk of SCD, COPD, more quickly and accurately. Using multiple diagnostics, e.g., orthogonal impedance measurements from two IMDs as described herein, may both improve specificity of determination of HF or other health condition status, as well as provide more context regarding the source of the patient condition. This also allows treatment to be provided and/or altered to a patient based on the detection more accurately and timely, which results in better treatment results for a patient suffering from such a health condition.

In contrast to clinic visit/stay based monitoring of a patient's condition and risk of adverse health events, the techniques of this disclosure may be implemented by systems including IMDs and computing devices that can autonomously and continuously (e.g., on a periodic or triggered basis without human intervention) measure impedance and sense/determine other patient parameter data while the IMD is implanted in a patient over months or years and perform numerous operations per second on the data to enable the systems herein to determine the health condition of the patient. Using techniques of this disclosure with IMDs may be advantageous when a physician cannot be continuously present with the patient over weeks or months to evaluate the parameters and/or where performing the operations on the data described herein (e.g., application of a machine learning model model) could not practically be performed in the mind of a physician. Using the techniques of this disclosure with autonomously/continuously operating IMDs and computing devices may provide a clinical advantage in timely detecting changes in a patient's condition and exercise tolerance threshold and providing timely alerts to the patient and/or caregiver.

In some examples, the techniques and systems of this disclosure may use a machine learning model to more accurately determine heart failure level or other health condition status based on impedance and other physiological data. In some examples, the machine learning model is trained with a set of training instances, where one or more of the training instances comprise data that indicate relationships between various input data and health condition statuses. Because the machine learning model is trained with potentially thousands or millions of training instances, the machine learning model may reduce the amount of error in determining health condition status, which may improve patient outcomes.

In one example, this disclosure describes a system for detecting statuses of health conditions of a patient includes a first implantable medical device (IMD) comprising one or more first sensors and configured to receive one or more first signals from the first sensors to determine one or more interstitial fluid index values based at least in part on the received first signals; and a second IMD comprising one or more second sensors and configured to receive one or more second signals from the second sensors to determine one or more intravascular fluid index values based at least in part on the received second signals; and processing circuitry configured to detect a health condition status of the patient based at least in part on the interstitial fluid index values and the intravascular fluid index values.

In another example, this disclosure describes a method of detecting statuses of health conditions of a patient includes determining one or more interstitial fluid index values based at least in part on one or more first signals received from a first IMD; determining one or more intravascular fluid index values based at least in part on one or more second signals received from a second IMD; and detecting a health condition status of the patient based at least in part on the interstitial fluid index values and the intravascular fluid index values.

In another example, this disclosure describes a non-transitory computer-readable storage medium comprising instructions that, when executed by processing circuitry, cause the processing circuitry to determine one or more interstitial fluid index values based at least in part on one or more first signals received from a first IMD; determine one or more intravascular fluid index values based at least in part on one or more second signals received from a second IMD; and detect a health condition status of the patient based at least in part on the interstitial fluid index values and the intravascular fluid index values, wherein the first signals include an impedance signal, and the second signals include an intracardiac impedance signal.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system configured to detect a health condition status of a patient in accordance with one or more techniques of this disclosure.

FIG. 2 is a block diagram illustrating an example configuration of a patient sensing device that operates in accordance with one or more techniques of the present disclosure.

FIG. 3 is block diagram illustrating an example configuration of a computing device that operates in accordance with one or more techniques of the present disclosure.

FIG. 4 is a block diagram illustrating an example configuration of a health monitoring system that operates in accordance with one or more techniques of the present disclosure.

FIG. 5 is a block diagram illustrating an example system that includes implantable medical device(s) used to obtain diagnostic states of various physiological parameters.

FIG. 6 is a flow diagram illustrating an example operation to detect a health condition status of a patient.

FIG. 7 is a flow diagram illustrating another example operation to detect a health condition status of a patient.

FIG. 8 is a conceptual diagram illustrating an example machine learning model configured to determine a health condition status of a patient based on subcutaneous and intravascular impedance.

FIG. 9 is a conceptual diagram illustrating an example training process for an artificial intelligence model, in accordance with examples of the current disclosure.

FIG. 10A is a perspective drawing illustrating an example IMD.

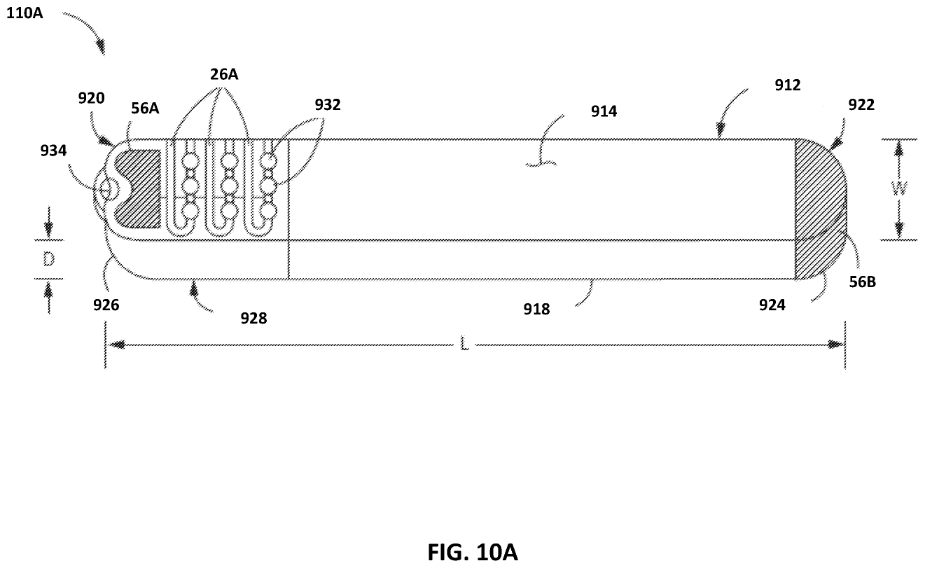

FIG. 10B is a perspective drawing illustrating another example IMD.

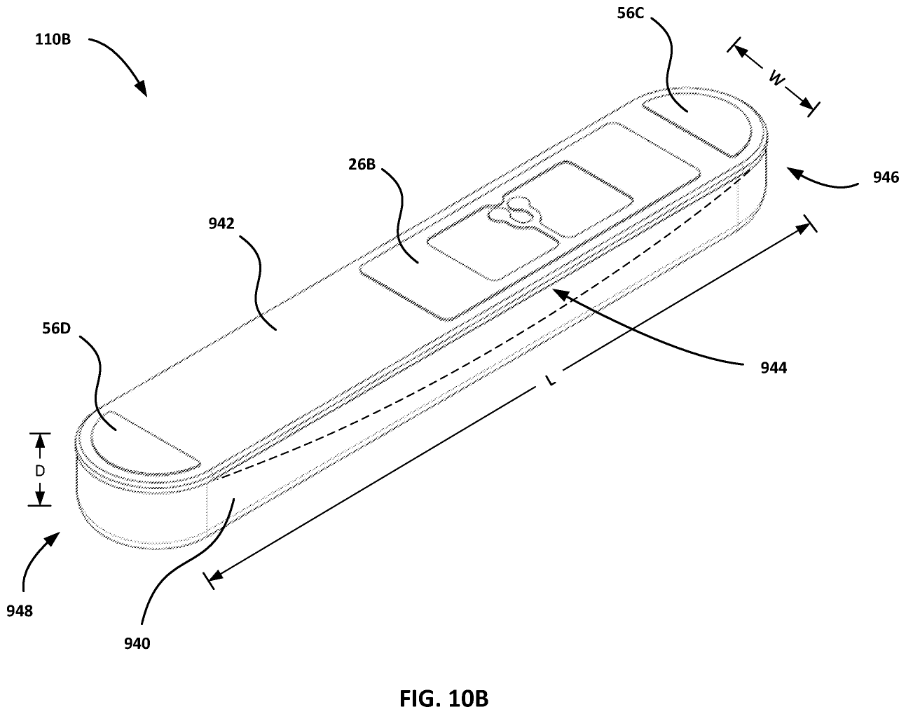

FIG. 11 is a conceptual diagram illustrating another example IMD.

Like reference characters refer to like elements throughout the figures and description.

### DETAILED DESCRIPTION

A variety of types of implantable and external devices are configured to detect a status of health conditions based on sensed physiological parameters. External devices that may be used to non-invasively sense and monitor physiological parameters include wearable devices with electrodes configured to contact the skin of the patient, such as patches, watches, rings, necklaces, hearing aids, clothing, car seats, or bed linens. Such external devices may facilitate relatively longer-term monitoring of patient health during normal daily activities.

Implantable medical devices (IMDs) also sense and monitor physiological parameters and detect status of health conditions such as HF, worsening HF, COPD, and risk of SCD. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic, Inc. of Minneapolis, Minnesota, which may be inserted subcutaneously. Another example of such an IMD is the Micra™ leadless pacemaker, available from Medtronic, Inc. Such IMDs may facilitate relatively longer-term monitoring of patients during normal daily activities, and may periodically transmit collected data to a remote patient monitoring system, such as the Medtronic Carelink™ Network.

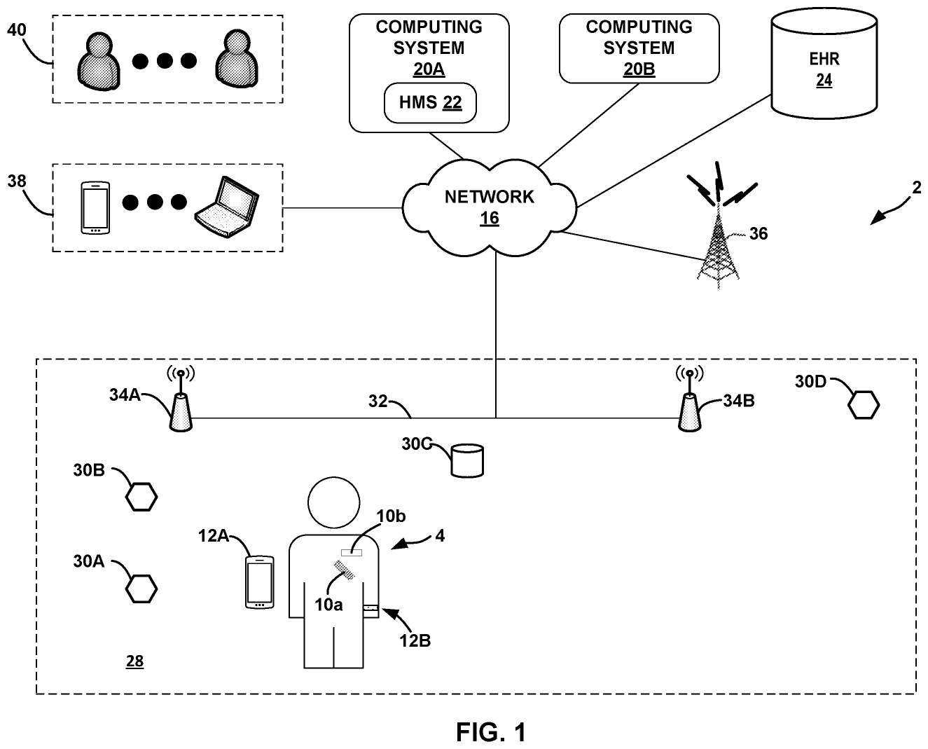

FIG. 1 is a block diagram illustrating an example system 2 configured detect statuses of health conditions of patient 4, and to respond to such detection, in accordance with one or more techniques of this disclosure. Patient 4 ordinarily, but not necessarily, will be a human. For example, patient 4 may be an animal needing ongoing monitoring for cardiac conditions. As used herein, the terms “detect,” “detection,” and the like may refer to detection of a status of a health condition presently (at the time or the period of time the data is collected) being experienced by patient 4, as well as detection based on the data that the condition of patient 4 is such that they have a suprathreshold likelihood of experiencing the health condition within a particular timeframe, e.g., prediction of the health condition. The example techniques may be used with two or more patient sensing devices, e.g., IMDs 10a and 10b (collectively, “IMDs 10”), which may be in wireless communication with each other and/or may be in wireless communication with one or more patient computing devices, e.g., patient computing devices 12A and 12B (collectively, “patient computing devices 12”). The two or more patient sensing devices 10 may be implanted within patient 4. In some examples, patient sensing devices may be external to (e.g., worn by) patient 4. For example, a system with two IMDs 10a, 10b or other sensing devices may capture different values of a common patient parameter with different resolution/accuracy based on their respective locations. Although not illustrated in FIG. 1, IMDs 10 may include electrodes and other sensors to sense physiological signals of patient 4, and may collect and store sensed physiological data based on the signals and detect a health condition status based on the data.

Patient computing devices 12 are configured for wireless communication with IMDs 10. Computing devices 12 retrieve sensed physiological data from IMDs 10 that was collected and stored by the IMDs 10. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMDs 10 such as a desktop, laptop, or tablet computer. Computing devices 12 may communicate with IMDs 10 and each other according to the Bluetooth® or Bluetooth® Low Energy (BLE) protocols, as examples. In some examples, only one of computing devices 12, e.g., computing device 12A, is configured for communication with IMDs 10, e.g., due to execution of software (e.g., part of a health monitoring application as described herein) enabling communication and interaction with one or more IMDs 10.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMDs 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 may facilitate detection of health condition status of patient 4 by system 2, and the responses of system 2 to detection of health condition status.

Computing device(s) 12 may transmit data, including data retrieved from IMDs 10 to computing system(s) 20 via network 16. The data may include sensed data, e.g., values of physiological parameters measured by IMDs 10 and, in some cases one or more of computing devices 12, data regarding health events detected by IMDs 10 and computing device(s) 12, and other physiological signals or data recorded by IMDs and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMDs 10 and/or computing devices 12 to detect statuses of health conditions for patient 4. In some examples, HMS 22 provides data from EHR 24 to computing device(s) 12 and/or IMDs 10 for storage therein and use as part of their algorithms for detecting statuses of health conditions.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

One or more of IMDs 10 may be configured to transmit data, such as sensed, measured, and/or determined values of physiological parameters (e.g., heart rates, impedance measurements, impedance scores, fluid indices, respiratory rate, activity data, cardiac ECGs, heart sounds, posture, QRS morphological features indicating fluid or electrolyte levels, historical physiological data, blood pressure values, etc.), to wireless access points 34 and/or computing device(s) 12. Wireless access points 34 and/or computing device(s) 12 may then communicate the retrieved data to computing systems 20 via network 16.

One or more of IMDs 10 may transmit data over a wired or wireless connection to computing system 20 or to computing device(s) 12. For example, computing device(s) 12 may receive data from IMDs 10 or from computing device(s) 12. In another example, computing device(s) 12 may receive data from computing system 20 or from medical IMDs 10, such as physiological parameter values, diagnostic states, or probability scores, via network 16. In such examples, computing device(s) 12 may determine the data received from computing system 20 or from IMDs 10 and may store the data to a storage device in the computing device(s) accordingly.

In addition, one or more of IMDs 10 may serve as or include data server(s). For example, IMDs 10 may include enough storage capacity or processing power to perform the techniques disclosed herein on a single one of IMDs 10 or on a network of IMDs 10 coordinating tasks via network 16, 32 (e.g., over a private or closed network). In some examples, one of IMDs 10 may include at least one of data server(s). For example, a portable/bedside patient monitor may be able to serve as a data server, as well as serving as one of IMDs 10a, 10b configured to obtain physiological parameter values from patient 4. In other examples, computing system 20 may communicate with each of IMDs 10 via a wired or wireless connection, to receive physiological parameter values or diagnostic states from IMDs 10. In some examples, physiological parameter values may be transferred from IMDs 10 to computing system 20 and/or to computing device(s) 12.

In some cases, computing system 20 may be configured to provide a secure storage site for data that has been collected from IMDs 10 and/or computing device(s) 12. In some instances, computing system 20 may include a database that stores medical- and health-related data. For example, computing system 20 may include a cloud server or other remote server that stores data collected from IMDs 10 and/or computing device(s) 12. In some cases, computing system 20 may assemble data in web pages or other documents for viewing by trained professionals, such as clinicians, via clinician computing devices 38. One or more aspects of the example system described with reference to FIG. 1 may be implemented with general network technology and functionality, which may be similar to that provided by the Medtronic CareLink® Network.

In some examples, one or more of clinician computing devices 38 may be a tablet or other smart device located with a clinician, by which the clinician may program, receive alerts from, and/or interrogate IMDs 10. For example, the clinician may access data collected by IMDs 10 through a clinician computing device 38, such as when patient 4 is in between clinician visits, to check on a status of a medical condition. In some examples, the clinician may enter instructions for a medical intervention for patient 4 into an application executed by clinician computing device 38, such as based on a status of a patient condition determined by IMDs 10, computing device(s) 12, computing system 20, or any combination thereof, or based on other patient data known to the clinician.

One clinician computing device 38 may transmit instructions for medical intervention to another of clinician computing devices 38 located with patient 4 or a caregiver of patient 4. For example, such instructions for medical intervention may include an instruction to change a drug dosage, timing, or selection, to schedule a visit with the clinician, or to seek medical attention. In further examples, a clinician computing device 38 may generate an alert to patient 4 (or relay an alert determined by IMDs 10, computing device(s) 12, or computing system 20) based on a probability score (e.g., posterior probability) determined from physiological parameter values of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

The splanchnic compartment, intravascular compartment, and interstitial compartment are three fluid compartments in a person such as patient 4. Fluid inside the intravascular compartment and interstitial compartment comprise what may be referred to as cardiac volume. In some examples, system 2 may determine the intravascular fluid and interstitial fluid levels with two separate devices to help detect a health condition status such as cardiac perfusion, intravascular volume, preload, vasoconstriction, vasodilation, fluid retention, and/or fluid redistribution. Example system 2 may be used to measure subcutaneous impedance and intracardiac impedance to provide to patient 4 and/or other users an early warning for the onset of HF, worsening HF, a HF decompensation event, COPD, and/or risk of SCD based on the orthogonal impedance measurements.

IMD 10a may be implanted outside of a thoracic cavity of patient 4 (e.g., subcutaneously in the pectoral location illustrated in FIG. 1. IMD 10a may be positioned near the sternum near or just below the level of the heart of patient 4, e.g., at least partially within the cardiac silhouette. In some examples, IMD 10a takes the form of the LINQ II™ ICM. IMD 10b may be implanted near or inside the heart. In some examples, IMD 10b takes the form of a pacemaker and may be inserted into the heart. In some examples, IMD 10b may be a leadless pacemaker. In some examples, IMD 10b takes the form of the Micra™. In some examples, IMD 10b takes the form of a transvenous implantable cardioverter defibrillator (ICD), cardiac resynchronization therapy (CRT) device, an implantable pulse generator (IPG), a subcutaneous ICD (S-ICD), or any other device to measure intracardiac pressures and/or impedances.

Although described primarily in the context of examples in which IMD 10a takes the form of an ICM and IMD 10b takes the form of a pacemaker, the techniques of this disclosure may be implemented in systems including any one or more implantable or external medical devices, including monitors, pacemakers, defibrillators, wearable external defibrillators, neurostimulators, or drug pumps. In some examples, instead of or in addition to two IMDs 10, system 2 may include a ventricular assist device or WAED in addition to IMD 10a, 10b.

In some examples, processing circuitry, e.g., processing circuitry 50 of IMDs 10, processing circuitry 130 of computing device(s) 12, or processing circuitry 23 of computing systems 20, may use absolute impedance values and statistical representations of impedance values when determining impedance scores. In some examples, absolute impedance values may generally refer to average impedance values. In some examples, processing circuitry 50 may determine whether the fluid index values and statistical representations of impedance values satisfy respective thresholds. As discussed herein, the thresholds may include adaptive thresholds. For example, processing circuitry 50 may compare the fluid index values to an adaptive threshold. In some examples, an adaptive threshold is determined based on the intra-day variation in impedance values and the absolute impedance. In any event, processing circuitry 50 may determine an impedance score that indicates a heart condition status of patient 4.

In some examples, IMD 10a may be configured to measure impedance values within the interstitial fluid of patient 4. For example, IMD 10a may be configured to receive one or more signals indicative of subcutaneous tissue impedance electrodes. In some examples, IMD 10a may be a purely diagnostic device. For example, IMD 10a may be a device that is to measure subcutaneous impedance values of patient 4. IMD 10a may determine fluid index values, such as interstitial fluid index value, using impedance signals received from electrodes. IMD 10a may also use the impedance value measurements to determine one or more interstitial fluid index values, impedance scores, and/or various thresholds, such as adaptive thresholds, scoring thresholds, weighting factors for thresholds, and/or cardiac risk thresholds. In some examples, other physiological parameters, such as posture, tissue perfusion, R-wave amplitude and width may also be used to determine one or more interstitial fluid index values.

Subcutaneous impedance may be measured by delivering a signal through an electrical path between electrodes (not shown in FIG. 1). In some examples, the housing of IMD 10a may be used as an electrode in combination with electrodes located on leads. For example, IMD 10a may measure subcutaneous impedance by creating an electrical path between a lead and one of the electrodes. In additional examples, IMD 10a may include an additional lead or lead segment having one or more electrodes positioned subcutaneously or within the subcutaneous layer for measuring subcutaneous impedance. In some examples, two or more electrodes useable for measuring subcutaneous impedance may be formed on or integral with the housing of IMD 10a. In some examples, one or more of the electrodes of IMD 10a contact interstitial fluid.

In some examples, IMD 10a may measure subcutaneous impedance of patient 4 and may process or send impedance data to aggregate evidence of decreasing impedance. The aggregated evidence is referred to as an interstitial fluid index and may be determined as function of the difference between measured impedance values and reference impedance values. The interstitial fluid index may then be used to determine an interstitial fluid level of patient 4 that may help indicate a heart condition status of patient 4.

In some examples, IMD 10a may also sense ECG signals via the plurality of electrodes and/or operate as a therapy delivery device. For example, IMD 10a may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

IMD 10b is configured to measure one or more of intracardiac impedance values and heart sounds, such as A1, A2, A3 and A4 (also referred to as S1, S2, S3 and S4), e.g., amplitude and slew measurements, that indicate intravascular fluid level of patient 4. For example, IMD 10b may be configured to receive one or more signals indicative of intracardiac impedance via intracardiac electrodes of IMD 10b. In some examples, IMD 10b may be a purely diagnostic device. For example, IMD 10b may be a device to measure intracardiac impedance values of patient 4. In some examples, IMD 10b may be configured to sense and generate one or more signals indicative of heart sounds. For example, IMD 10b may be configured to measure heart sounds. IMD 10b may determine fluid index values, such as intravascular fluid index value, using impedance signals received from electrodes and/or measured heart sounds. IMD 10b may also use one or more of the impedance value measurements and the heart sound measurements to determine one or more intravascular fluid index values, impedance scores, and/or various thresholds, such as adaptive thresholds, scoring thresholds, weighting factors for thresholds, and/or cardiac risk thresholds. In some examples, other physiological parameters, such as posture, tissue perfusion, R-wave amplitude and width may also be used to determine one or more intravascular fluid index values.

Intracardiac impedance may be measured by delivering a signal through an electrical path between electrodes (not shown in FIG. 1). In some examples, the housing of IMD 10b may be used as an electrode in combination with electrodes located on leads. For example, IMD 10b may measure intracardiac impedance by creating an electrical path between a lead and one of the electrodes. In additional examples, IMD 10b may include an additional lead or lead segment having one or more electrodes positioned intravascularly or within the subcutaneous layer for measuring intracardiac impedance. In some examples, IMD 10b may include intracardiac leads to measure an intracardiac impedance vector. In some examples, two or more electrodes useable for measuring intracardiac impedance may be formed on or integral with the housing of IMD 10b.

In some examples, IMD 10b may measure intracardiac impedance of patient 4 and may process or send impedance data to aggregate evidence of decreasing impedance. The aggregated evidence is referred to as an intravascular fluid index and may be determined as function of the difference between measured impedance values and reference impedance values. The intravascular fluid index may then be used to determine an intravascular fluid level of patient 4 that may help indicate a heart condition status of patient 4.

In some examples, IMD 10b may include intracardiac pressure sensors to measure one or more pressures indicative of an intravascular fluid level. In some examples, IMD 10b may be a purely diagnostic device. For example, IMD 10b may be a device that only measures intracardiac impedance values of patient 4. In some examples, IMD 10b may also use one or more of the impedance value measurements, heart sound measurements, and/or pressure measurements to determine one or more intravascular fluid index values, impedance scores, and/or various thresholds, such as adaptive thresholds, scoring thresholds, weighting factors for thresholds, and/or cardiac risk thresholds.

In some examples, IMD 10b may also sense ECG signals via the plurality of electrodes and/or operate as a therapy delivery device. For example, IMD 10b may additionally operate as a therapy delivery device to deliver electrical signals to the heart of patient 4, such as an implantable pacemaker, a cardioverter, defibrillator, a drug delivery device that delivers therapeutic substances to patient 4 via one or more catheters, and/or as a combination therapy device that delivers both electrical signals and therapeutic substances. In some examples, IMD 10b may alter therapy to patient 4 based on information about the fluid distribution. For example, IMD 10b may increase pacing to increase heart rate of patient 4 based on the determined intravascular fluid level and interstitial fluid level. In some examples, IMD 10b may alter delivery of therapeutic substances to patient 4 based on the determined intravascular fluid level and interstitial fluid level.

In some examples, when a patient is undergoing worsening HF, the patient may retain fluid which may result in the patient gaining weight. Accordingly, a patient's weight gain may be monitored as an indication of fluid retention. In some examples, when a patient is undergoing worsening HF, the patient may redistribute fluid to the intravascular space and/or interstitial space, which may result in little to no weight gain by a patient. The interstitial fluid levels and the intravascular fluid levels, how they change over a specific period of time, and how they relate to each other may provide more accurate and more efficient indications of HF, worsening HF, COPD, and/or risk of SCD.

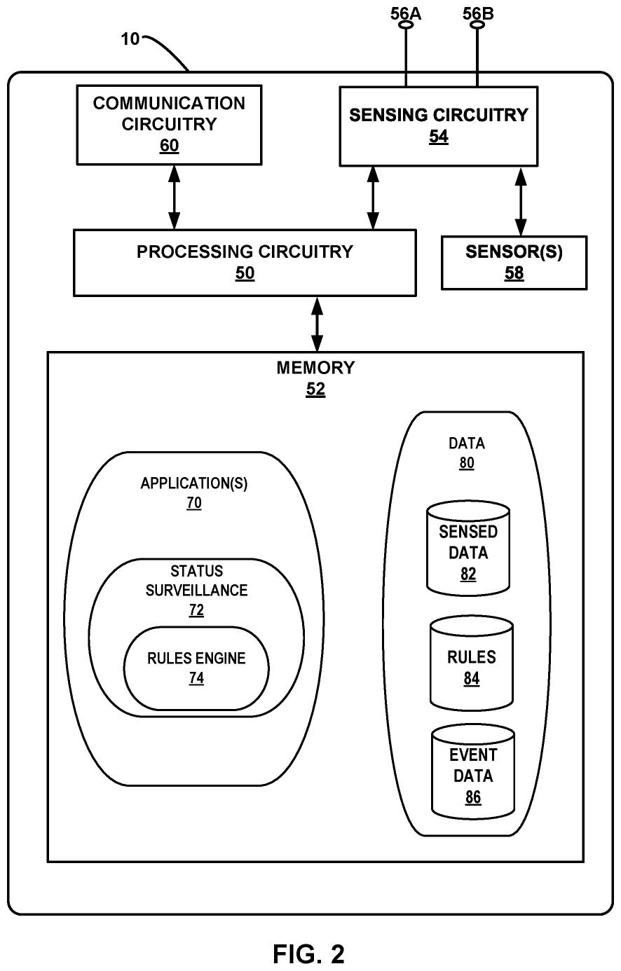

IMDs 10a, 10b may sense physiological parameters to determine interstitial fluid level and intravascular fluid level, as discussed above. An interstitial fluid level corresponds to an amount of fluid in an interstitial reservoir in patient 4. An intravascular fluid level corresponds to an amount of fluid in an intravascular reservoir in patient 4. As shown in FIGS. 2, 3, and 5, processing circuitry 50, 130, 23 is located in respective IMD 10a, IMD 10b, patient computing devices 12, and computing systems 20. For simplicity, processing circuitry 50 will be referenced in the examples discussed below, but any one or more of processing circuitries 50, 130, and 23 may be used as the processing circuitry. Processing circuitry 50 may determine an interstitial fluid index value based on the sensed impedance signals of IMD 10a and determine an intravascular fluid index value based on the sensed impedance signals and/or the heart sound measurements of IMDs 10b. Processing circuitry 50 may determine interstitial fluid levels based on a determined interstitial fluid index value which may be based in part on a signal received from IMD 10a. In some examples, the signal received from IMD 10a is based on a measured subcutaneous impedance. Processing circuitry 50 may determine intravascular fluid levels based on a determined intravascular fluid index which may be based in part on a signal received from IMD 10b. In some examples, the signal received from IMD 10b is based on a measured intracardiac impedance. In some examples, the signal received from IMD 10b is based on a measured heart sounds.

In some examples, IMD 10a may obtain sensor data every few minutes, hourly, or even daily. In some examples, the sensor data is obtained every few minutes, such as five minutes, and the processing circuitry 50 may determine the interstitial fluid index values based on sensor data aggregated over a period of time, such as an hour, or a day. IMD 10a may aggregate the interstitial fluid index values based on the sensor data from IMD 10a over a period of time, such as an hour, and determine an hourly average. Processing circuitry 50 may aggregate the interstitial fluid index values over a period of time and determine an interstitial fluid level based at least in part on the aggregated interstitial fluid index values.

The processing circuitry 50 may aggregate the hourly measurements, while removing noisy measurements, to determine daily measurements that indicate interstitial fluid indexes which correspond to interstitial fluid levels. Processing circuitry 50 may aggregate a plurality of interstitial fluid levels for an average fluid value for a period of time, such as an hour, day, or week.

In some examples, IMD 10b may obtain sensor data every few minutes, hourly, or even daily. In some examples, the sensor data is obtained every few minutes, such as five minutes, and the processing circuitry may determine intravascular fluid index values based on sensor data aggregated over a period of time, such as an hour, or a day. IMD 10b may aggregate the intravascular fluid index values based on the sensor data from IMD 10b over a period of time, such as an hour, and determine an hourly average. Processing circuitry 50 may aggregate the intravascular fluid index values over a period of time and determine an intravascular fluid level based at least in part on the aggregated intravascular fluid index values.

In some examples, IMD 10a and/or IMD 10b may determine fluid index values in accordance with U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, and/or in accordance with U.S. application Ser. No. 17/021,489 by Sarkar et al., entitled “DETERMINING HEART CONDITION STATUSES USING SUBCUTANEOUS IMPEDANCE MEASUREMENTS”, all of which are incorporated herein by reference in their entirety.

Processing circuitry 50 may aggregate the hourly measurements, while removing noisy measurements, to determine daily measurements that indicate intravascular fluid indexes which correspond to intravascular fluid levels. Processing circuitry may aggregate a plurality of intravascular fluid levels. Processing circuitry 50 may analyze changes in determined intravascular fluid levels and changes in determined interstitial fluid levels over a period of time, such as hours, days, or weeks, and detect a status of a health condition based at least in part on one or more of change of intravascular fluid level over a period of time and change of interstitial fluid level over a period of time.

In some examples, processing circuitry 50 may compare the changes in intravascular fluid levels over a period of time to the changes of the interstitial fluid levels over a period of time and detect a health condition status based at least in part on the comparison. In some examples, processing circuitry 50 may detect a health condition status based at least in part on one or more of changes in the interstitial fluid levels over a period of time, changes in the intravascular levels over a period of time, the intravascular fluid level at a particular time, and the interstitial fluid level at the particular time.

In some examples, processing circuitry 50 may compare the determined interstitial fluid levels and the intravascular fluid levels to detect a status of a health condition. In some examples, processing circuitry 50 may detect a status of a health condition based on the comparison of the interstitial fluid levels and the intravascular fluid levels and additional other measured diagnostic parameters. In some examples, processing circuitry 50 may compare the changes in intravascular fluid levels over a period of time to the changes of the interstitial fluid levels over a period of time and detect a health condition status based at least in part on the comparison. In some examples, processing circuitry 50 may detect a health condition status based at least in part on one or more of changes in the interstitial fluid levels over a period of time, changes in the intravascular levels over a period of time, the intravascular fluid level at a particular time, and the interstitial fluid level at the particular time.

In some examples, patient 4 may be determined to be vasodilated when the intravascular fluid levels are greater than the interstitial fluid levels by an amount greater than a threshold level. In some examples, patient 4 may be determined to be vasoconstricted and less perfuse when the intravascular fluid levels are less than the interstitial fluid levels by an amount greater than a threshold.

In some examples, a health condition may be HF, worsening HF, COPD, and risk of SCD. In some examples, processing circuitry 50 may determine an increased risk of left heart failure in response to determining the intravascular fluid level has increased above a threshold and the interstitial level has increased above a threshold. In some examples, processing circuitry 50 may determine an increased risk of right heart failure in response to determining the interstitial fluid level has increased above a threshold while and an increase in intravascular fluid level is below a threshold. In some examples, timing in which fluid levels change may indicate a health condition. In some examples, the differences in the rates of change of the fluid levels being compared to a threshold may indicate a type of heart failure. For example, the interstitial fluid level increasing at a greater rate than the intravascular fluid level may indicate right heart failure. In some examples, the intravascular fluid increasing at a greater rate than the interstitial fluid level may indicate left heart rate failure. In some examples, the intravascular fluid increasing above a threshold before the interstitial fluid level increases above a threshold may indicate left heart failure.

In fluid retention, both the interstitial fluid level and the intravascular fluid level increase. In fluid redistribution, the interstitial fluid level increase while the intravascular fluid level maintains a same level or increases slightly. Processing circuitry 50 may determine fluid retention has occurred when determining the intravascular fluid level has increased above a threshold and the interstitial level has increased above a threshold. Processing circuitry 50 may determine redistribution has occurred when determining the interstitial fluid level has increased above a threshold while and an increase in intravascular fluid level is below a threshold. In some examples, timing in which fluid levels change may indicate fluid retention. In some examples, the differences in the rates of change of the fluid levels being compared to a threshold may indicate fluid retention.

A therapy may be determined based on the determined interstitial fluid levels and the intravascular fluid levels. In some examples, a nitrate therapy may be instructed instead of diuretic therapy if the determined fluid levels indicate patient 4 is not vasodilated. In some examples, processing circuitry 50 may determine whether nitrates may be provided in addition to diuretics based on the determined interstitial fluid levels and intravascular fluid levels in conjunction with an A2 slew value that may be measured by IMD 10b and/or pulse pressure waveforms that may be measured by IMD 10a.

In some examples, after processing circuitry 50 detects a health condition status of the patient, as discussed above, IMD 10b may perform treatment in response to the detected health condition status, such as generating electrical impulses to provide pacing therapy, based on the detected health condition status, to cause a heart to contract. IMD 10b may perform one or more various other treatments based on the detected health condition status.

In some examples, after processing circuitry 50 detects a health condition status of the patient, the detected health condition status may be displayed and/or transmitted to another computing device, such as clinician computing device 38, to inform a clinician of the health condition status of patient 4. In some examples, after processing circuitry 50 detects a health condition status of the patient, the detected health condition status may be transmitted to be stored in EHR 24.

As described above, system 2 may be configured to detect health condition status of patient 4, such as HF, worsening HF, COPD, and risk of SCD, based on data sensed by IMDs 10a, 10b and, in some cases, other data, such as data sensed by computing devices 12A and/or 12B, and data from EHR 24. To detect a status of a health condition, IMDs 10, computing devices 12, and/or computing systems 20 may apply rules to the data, which may be referred to as patient parameter data. In response to detection of a status of a health condition, IMDs 10, computing devices 12, and/or computing systems 20 may wirelessly transmit a message to one or both of computing devices 12 and/or to a clinician computing device 38. The message may indicate the detected health condition status of the patient. The message may indicate a time that IMDs 10 detected the health condition status.

The message may include physiological data collected by IMDs 10, e.g., data which lead to detection of the health condition status, data prior to detection of the health condition status, and/or real-time or more recent data collected after detection of the health condition status. The physiological data may include values of one or more physiological parameters and/or digitized physiological signals such as subcutaneous impedance, intracardiac impedance, interstitial fluid index, intravascular fluid index, interstitial fluid level, and/or intravascular fluid level, as well as other physiological parameters. Examples of statuses of health conditions are HF, worsening HF, COPD, risk of SCD, a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

As illustrated in FIG. 1, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, televisions, lights, locks, thermostats, appliances, actuators, controllers, or any other smart home (or building) devices. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take actions. In some examples, IMDs 10 are configured to communicate wirelessly with one or more of IoT devices 30, e.g., in response to detection of a health condition status when communication with computing devices 12 is unavailable. In such examples, IoT device(s) 30 may be configured to provide some or all of the functionality ascribed to computing devices 12 herein.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally or alternatively, e.g., when local network is unavailable, computing devices 12, IoT devices 30, and other devices within environment 28 may be configured to communicate with network 16, e.g., with HMS 22, via a cellular base station 36 and a cellular network.

In some examples, computing device(s) 12 and/or computing system 20 may implement one or more algorithms to evaluate the sensed physiological data received from IMDs 10. In some examples, computing device(s) 12 and/or computing system 20 may have greater processing capacity than IMDs 10, enabling more complex analysis of the data. In some examples, the computing device(s) 12 and/or computing system 20 may apply the data to a machine learning model or other artificial intelligence developed algorithm, e.g., to determine whether the data is sufficiently indicative of the health condition status.

In some examples, HMS 22 may be configured to transmit messages to one or computing devices 38 associated with one or more clinicians 40 via network 16. Care providers may include emergency medical systems (EMS) and hospitals, and may include particular departments within a hospital, such as an emergency department, a cardiology department, a catheterization lab, or a stroke response department. Clinician computing devices 38 may include smartphones, desktop, laptop, or tablet computers, or workstations associated with such systems or entities, or employees of such systems or entities. The messages may include any of the data collected by IMDs 10, computing device(s) 12, and IoT device(s) 30, including sensed physiological data, time of the health condition status, location of patient 4, and results of the analysis by IMDs 10, computing device(s) 12, IoT device(s) 30, and/or HMS 22. The information transmitted from HMS 22 to clinicians 40 may improve the timeliness and effectiveness of treatment for a health condition of patient 4 by clinicians 40.

FIG. 2 is a block diagram illustrating an example configuration of IMDs 10 of FIG. 1. IMD 10 may be an example of IMD 10a and/or IMD 10b but will be referenced below as IMD 10 for simplicity. As shown in FIG. 2, IMD 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 53 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may measure impedance, e.g., of tissue proximate to IMD 10, via electrodes 56. The measured impedance may vary based on respiration, fluid retention, cardiac pulse or flow, and a degree of perfusion or edema. Processing circuitry 50 may determine physiological data relating to respiration, fluid retention, cardiac pulse or flow, perfusion, and/or edema based on the measured impedance.

Sensing circuitry 54 may also monitor signals from electrodes 56 in order to, for example, monitor electrical activity of a heart of patient 4 and produce sensor data for patient 4. In some examples, processing circuitry 50 may identify features of the sensed ECG, such as heart rate, heart rate variability, T-wave alternans, intra-beat intervals (e.g., QT intervals), and/or ECG morphologic features, such as R-wave amplitude, max slew and width, and T-wave morphology, amplitude and slew, to detect an episode of cardiac arrhythmia of patient 4.

In some examples, IMD 10 includes one or more sensors 58, such as one or more accelerometers, gyroscopes, microphones, optical sensors, temperature sensors, pressure sensors, and/or chemical sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52. Patient parameters determined from signals from sensors 58 may include intravascular fluid level, interstitial fluid level, oxygen saturation, glucose level, stress hormone level, heart sounds, body motion, body posture, or blood pressure.

Memory 52 may store applications 70 executable by processing circuitry 50, and data 80. Applications 70 may include a health condition status surveillance application 72. Processing circuitry 50 may execute status surveillance application 72 to detect a health condition status of patient 4 based on combination of one or more of the types of physiological data from IMD 10a and/or IMD 10b, described herein, which may be stored as sensed data 82. In some examples, sensed data 82 may additionally include patient parameter data sensed by other devices, e.g., computing device(s) 12 or IoT device(s) 30, and received via communication circuitry 60. Status surveillance application 72 may be configured with a rules engine 74. Rules engine 74 may apply rules 84 to sensed data 82. Rules 84 may include one or more models, algorithms, decision trees, and/or thresholds. In some cases, rules 84 may be developed based on machine learning, e.g., may include one or more machine learning models.

As examples, status surveillance application 72 may detect HF, worsening HF, COPD, risk of SCD, sudden cardiac arrest (SCA), a ventricular fibrillation, a ventricular tachycardia, supra-ventricular tachycardia (e.g., conducted atrial fibrillation), ventricular asystole, or a myocardial infarction based on an ECG and/or other patient parameter data indicating the electrical or mechanical activity of the heart of patient 4. In some examples, status surveillance application 72 may detect stroke based on such cardiac activity data. In some examples, sensing circuitry 54 may detect brain activity data, e.g., an electroencephalogram (EEG) via electrodes 56, and status surveillance application 72 may detect stroke or a seizure based on the brain activity alone, or in combination with cardiac activity data or other physiological data. In some examples, status surveillance application 72 detects whether the patient has fallen based on data from an accelerometer alone, or in combination with other physiological data. When status surveillance application 72 detects a status of a health condition, status surveillance application 72 may store the sensed data 82 that lead to the detection (and in some cases a window of data preceding and/or following the detection) as event data 86.

In some examples, in response to detection of a health condition status, processing circuitry 50 transmits, via communication circuitry 60, event data 86 for the event to computing device(s) 12 (FIG. 1). This transmission may be included in a message indicating the health condition status, as described herein. Transmission of the message may occur on an ad hoc basis and as quickly as possible. Communication circuitry 60 may include any suitable hardware, firmware, software, or any combination thereof for wirelessly communicating with another device, such as computing devices 12, other IMD 10, and/or IoT devices 30.

In some examples, communication circuitry 60 in IMD 10a may communicate with IMD 10b to share information such as sensed data 82 and/or event data 86 to be used in the detecting a status of a health condition of patient 4. In some examples, IMD 10b may also communicate with IMD 10A to share information such as sensed data 82 and/or event data 86 to be used in the detecting a status of a health condition of patient 4.

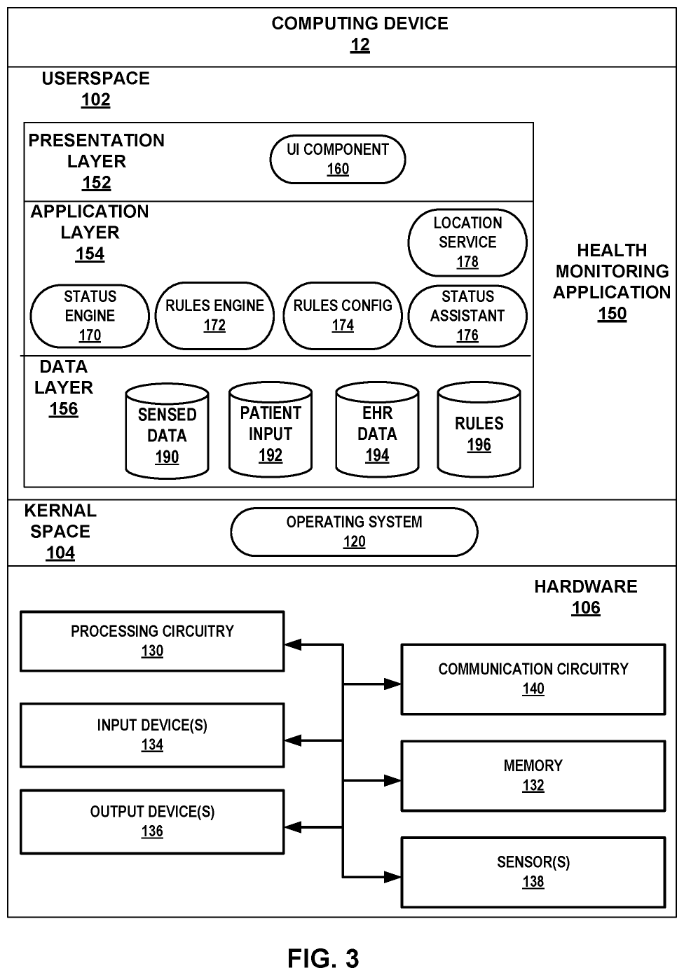

FIG. 3 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 and/or computing devices 38 and 42 may be configured similarly to the configuration of computing device 12 illustrated in FIG. 3.

As shown in the example of FIG. 3, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 3, hardware 106 includes processing circuitry 130, memory 132, one or more input devices 134, one or more output devices 136, one or more sensors 138, and communication circuitry 140. Although shown in FIG. 3 as a stand-alone device for purposes of example, computing device 12 may be any component or system that includes processing circuitry or other suitable computing environment for executing software instructions and, for example, need not necessarily include one or more elements shown in FIG. 3.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g. including non-volatile storage elements. Examples of such non-volatile storage elements include magnetic hard discs, optical discs, floppy discs, flash memories, or forms of electrically programmable memories (EPROM) or electrically erasable and programmable (EEPROM) memories. In some examples, memory 132 includes cloud-associated storage.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, haptic, audio, and visual output. Output devices 134 of computing device 12 may include a presence-sensitive screen, sound card, video graphics adapter card, speaker, cathode ray tube monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, accelerometers (e.g., 3-axis accelerometers), an optical sensor, impedance sensors, temperature sensors, pressure sensors, heart sound sensors (e.g., microphones), and other sensors, and sensing circuitry (e.g., including an ADC), similar to those described above with respect to IMDs 10 and FIG. 2.

Communication circuitry 140 of computing device 12 may communicate with other devices by transmitting and receiving data. Communication circuitry 140 may receive data from IMDs 10, such as subcutaneous impedance and intracardiac impedance, from communication circuitry in IMDs 10. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 3, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, status engine 170, rules engine 172, rules configuration component 174, status assistant 176, and location service 178. Status engine 170 may be responsive to receipt of a transmission from IMDs 10 indicating that sensed data 190 from IMDs 10 has been received and begin a determination of a health condition status of patient 4. Status engine 170 may also control performance of any of the operations in response to detection of a health condition status ascribed herein to computing device 12, such as transmitting messages to HMS 22 and controlling IoT devices 30.

Rules engine 174 analyzes sensed data 190, and in some examples, patient input 192 and/or EHR data 194, to determine an intravascular fluid index values and interstitial fluid index values and/or intravascular fluid levels and interstitial fluid values to detect a health condition status of patient 4. Sensed data 190 may include data received from IMDs 10, such as subcutaneous impedance and intracardiac impedance, as part of the transmission, additional data transmitted from IMDs 10, e.g., in “real-time,” and physiological and other data related to the condition of patient 4 collected by, for example, computing device(s) 12 and/or IoT devices 30. Sensed data 190 from IMDs 10 may further include determined intravascular fluid index values and interstitial fluid index values. Sensed data 190 may further include additional sensed data than received from IMDs 10. As examples sensed data 190 from computing device(s) 12 may include one or more of: activity levels, walking/running distance, 6-minute walking distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user. The queries and responses may occur responsive to the detection of the health condition status by IMDs 10 or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of SCA, tachyarrhythmia, myocardial infarction, stroke, seizure, one or more disease states, such as status of heart failure, COPD, renal dysfunction, or hypertension, aspects of disease state, such as ECG characteristics, cardiac ischemia, oxygen saturation, lung fluid, activity, or metabolite level, genetic conditions, congenital anomalies, history of procedures, such as ablation or cardioversion, and healthcare utilization. EHR data 194 may also include cardiac indicators, such as ejection fraction and left-ventricular wall thickness. EHR data 194 may also include demographic and other information of patient 4, such as age, gender, race, height, weight, and BMI.

Rules engine 172 may apply rules 196 to the data. Rules 196 may include one or more models, algorithms, decision trees, and/or thresholds. The rules 196 may include any of the rules discussed above with respect to a relationship of interstitial fluid levels and intravascular fluid levels to determining a health condition status. The rules 196 may further include rules applied to additional parameters measured by any of the sensors discussed above.

In some cases, rules 196 may be developed based on machine learning, e.g., may include one or more machine learning models. In some examples, rules 196 and the operation of rules engine 172 may provide a more complex analysis the patient parameter data, e.g., the data received from IMDs 10, than is provided by rules engine 74 and rules 84. In examples in which rules 196 include one or more machine learning models, rules engine 172 may apply feature vectors derived from the data to the model(s).

Rules configuration component 174 may be configured to modify rules 196 (and in some examples rules 84) based on feedback indicating whether the detections and confirmations of statuses of health conditions by IMDs 10 and/or computing device 12 were accurate. The feedback may be received from patient 4, or from clinicians 40 and/or EHR 24 via HMS 22. In some examples, rules configuration component 174 may utilize the data sets from true and false detections for supervised machine learning to further train models included as part of rules 196.

Rules configuration component 174, or another component executed by processing circuitry of system 2, may select a configuration of rules 196 based on etiological data for patient, e.g., any combination of one or more of the examples of sensed data 190, patient input 192, and EHR data 194 discussed above. In some examples, different sets of rules 196 tailored to different cohorts of patients may be available for selection for patient 4 based on such etiological data.

As discussed above, status assistant 176 may provide a conversational interface for patient 4 to exchange information with computing device 12. Responses from the user may be included as patient input 192. Status assistant 176 may use natural language processing and context data to interpret utterances by the user. In some examples, in addition to receiving responses to queries posed by the assistant, status assistant 176 may be configured to respond to queries posed by the user. In some examples, status assistant 176 may provide directions to and respond to queries regarding treatment of patient 4.

Location service 178 may determine the location of computing device 12 and, thereby, the presumed location of patient 4. Location service 178 may use global position system (GPS) data, multilateration, and/or any other known techniques for locating computing devices.

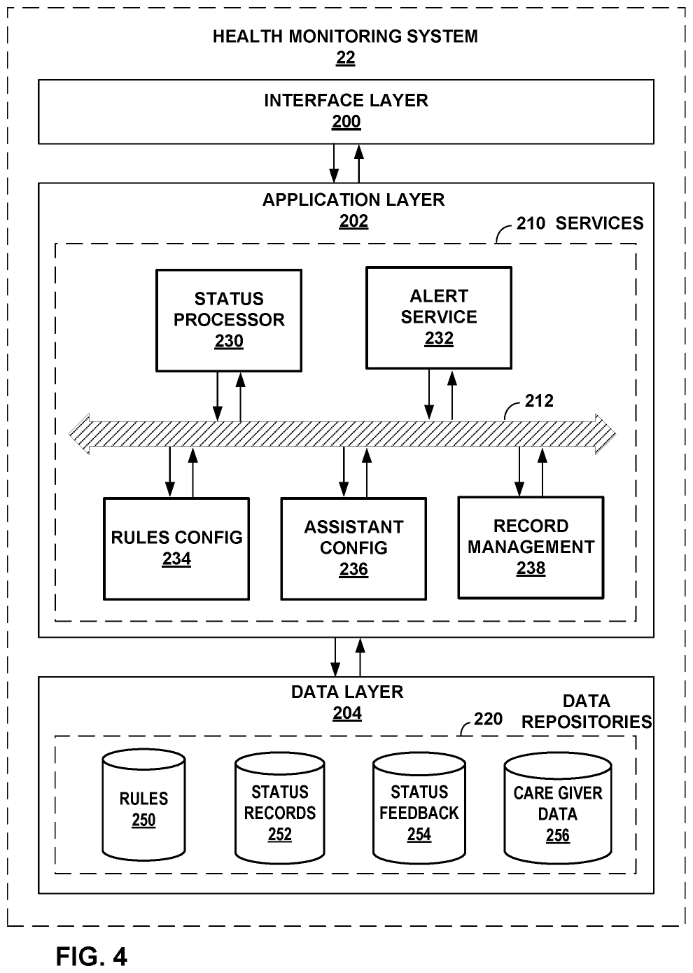

FIG. 4 is a block diagram illustrating an operating perspective of HMS 22.

HMS 22 may be implemented in a computing system 20, which may include hardware components such as those of computing device 12, e.g., processing circuitry, memory, and communication circuitry, embodied in one or more physical devices. FIG. 4 provides an operating perspective of HMS 22 when hosted as a cloud-based platform. In the example of FIG. 4, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 4, HMS 22 also includes an application layer 202 that represents a collection of services 210 for implementing the functionality ascribed to HMS herein. Application layer 202 receives information from client applications, e.g., sensed data from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 4, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Status processor service 230 may be responsive to receipt of sensed data, fluid index values, and/or detected health condition status from computing device(s) 12 and/or IoT device(s) 30 indicating that IMDs 10 sensed and transmitted data and, in some examples, detected a health condition status of patient 4. Status processor service 230 may initiate performance of any of the operations in response to receipt of sensed data, fluid index values, and/or detected health condition status ascribed herein to HMS 22, such as analyzing data to detect health condition status of patient 4 and, in some cases, communicating with patient 4 and clinicians 40.

Record management service 238 may store the patient data included in a received alert message within status records 252. Alert service 232 may package the some or all of the data from the status record, in some cases with additional information as described herein, into one or more alert messages for transmission to clinicians 40. Care giver data 256 may store data used by alert service 232 to identify to whom to send alerts based on applicability of the care provided by clinicians 40 to the health condition status of patient 4.

In examples in which HMS 22 performs an analysis to determine a health condition status of patient 4, status processor service 230 may apply one or more rules 250 to the sensed data received in the message, e.g., to feature vectors derived by status processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN),
Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to determine a health condition status, rules 250 maintained by HMS 22 may include rules 196 utilized by computing devices 12 and rules 84 used by IMD 10. In such examples, rules configuration service 250 may be configured to develop and maintain rules 196 and rules 84. Rules configuration service 234 may be configured to develop different sets of rules 84, 196, 250, e.g., different machine learning models, for different cohorts of patients. Rules configuration service 234 may be configured to modify these rules based on status feedback data 254 that indicates whether the detections and confirmations of statuses of health conditions by IMDs 10, computing device 12, and/or HMS 22 were accurate. Status feedback 254 may be received from patient 4, e.g., via computing device(s) 12, or from clinicians 40 and/or EHR 24. In some examples, rules configuration service 234 may utilize status records from true and false detections (as indicated by status feedback data 254) and confirmations for supervised machine learning to further train models included as part of rules 250.

As illustrated in the example of FIG. 4, services 210 may also include an assistant configuration service 236 for configuring and interacting with status assistant 176 implemented in computing device 12 or other computing devices. For example, assistant configuration service 236 may provide status assistants updates to their natural language processing and context analyses to improve their operation over time. In some examples, assistant configuration service 236 may apply machine learning techniques to analyze sensed data and status assistant interactions stored in status records 252, as well as the ultimate disposition of the status, e.g., indicated by EHR 24, to modify the operation of status assistants, e.g., for patient 4, a class of patients, all patients, or for particular users or devices, e.g., care givers, etc.

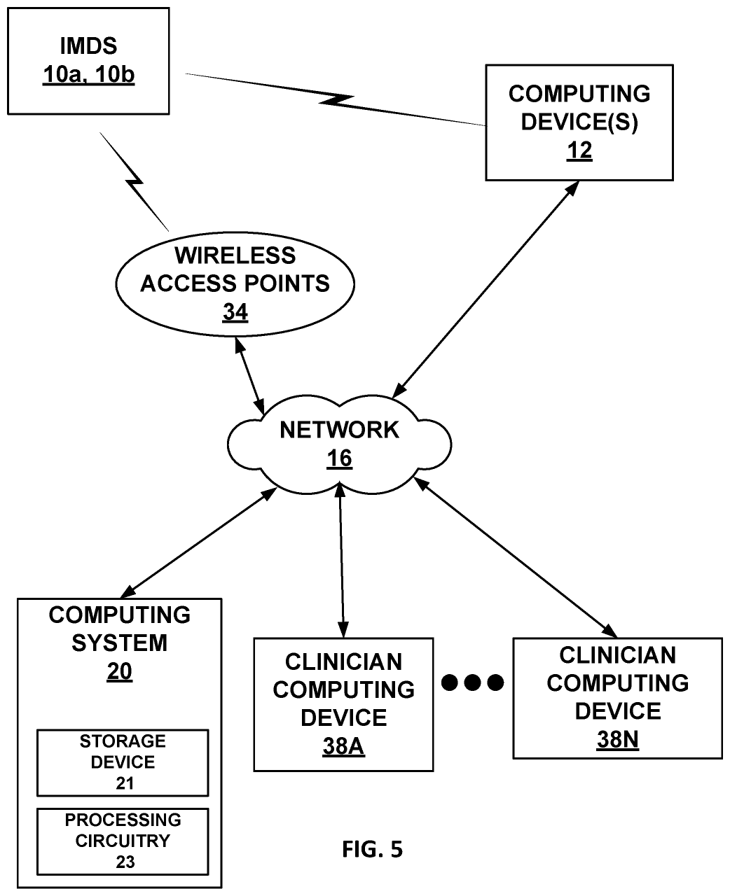

FIG. 5 is a block diagram illustrating an example system that includes wireless access points 34, a network 16, external computing devices, such as computing systems 20, and one or more other clinician computing devices 38A-38N (collectively, “clinician computing devices 38”), which may be coupled to IMDs 10 and computing device(s) 12 via network 16, in accordance with one or more techniques described herein. In this example, IMDs 10 may use communication circuitry 60 to communicate with computing device(s) 12 via a first wireless connection, and to communicate with wireless access points 34 via a second wireless connection. In the example of FIG. 5, wireless access points 34, computing device(s) 12, computing systems 20, and clinician computing devices 38 are interconnected and may communicate with each other through network 16. Network 16 may include a local area network, wide area network, or global network, such as the Internet. The system of FIG. 5 may be implemented, in some aspects, with general network technology and functionality similar to that provided by the Medtronic CareLink® Network.

Wireless access points 34 may include a device that connects to network 16 via any of a variety of connections, such as telephone dial-up, digital subscriber line (DSL), or cable modem connections. In other examples, wireless access points 34 may be coupled to network 16 through different forms of connections, including wired or wireless connections. In some examples, wireless access points 34 may be a user device, such as a tablet or smartphone, that may be co-located with the patient. IMD 10 may be configured to transmit data, such as impedance value information, impedance scores, and/or ECGs, to wireless access points 34. Wireless access points 34 may then communicate the retrieved data to computing systems 20 via network 16.

In some cases, computing systems 20 may be configured to provide a secure storage site for data that has been collected from IMDs 10 and/or computing device(s) 12. In some cases, computing systems 20 may assemble data in web pages or other documents for viewing by trained professionals, such as clinicians, via clinician computing devices 38. One or more aspects of the illustrated system of FIG. 5 may be implemented with general network technology and functionality, which may be similar to that provided by the Medtronic CareLink® Network.

In some examples, computing systems 20 may monitor impedance, e.g., based on measured impedance information received from IMDs 10 and/or computing device(s) 12 via network 16, to detect worsening heart failure of patient 4 using any of the techniques described herein. Computing systems 20 may provide alerts relating to worsening heart failure of patient 4 via network 16 to patient 4 via wireless access points 34, or to one or more clinicians via computing devices 100. In examples such as those described above in which IMD 10 and/or computing device(s) 12 monitor the impedance, computing systems 20 may receive an alert from IMDs 10 or computing device(s) 12 via network 16, and provide alerts to one or more clinicians via clinician computing devices 38. In some examples, computing systems 20 may generate web-pages to provide alerts and information regarding the impedance, and may include a memory to store alerts and diagnostic or physiological parameter information for a plurality of patients.

In some examples, one or more of clinician computing devices 38 may be a tablet or other smart device located with a clinician, by which the clinician may program, receive alerts from, and/or interrogate IMDs 10. For example, the clinician may access data collected by IMDs 10 through a clinician computing device 38, such as when patient 4 is in between clinician visits, to check on a status of a medical condition. In some examples, the clinician may enter instructions for a medical intervention for patient 4 into an application executed by clinician computing device 38, such as based on a status of a patient condition determined by IMDs 10, computing device(s) 12, computing systems 20, or any combination thereof, or based on other patient data known to the clinician. Clinician computing device 100 then may transmit the instructions for medical intervention to another of clinician computing devices 100 located with patient 4 or a caregiver of patient 4.

In some examples, instructions for medical intervention may include an instruction to change a drug dosage, timing, or selection, to schedule a visit with the clinician, or to seek medical attention. In further examples, a clinician computing device 38 may generate an alert to patient 4 based on a status of a medical condition of patient 4, which may enable patient 4 to proactively seek medical attention prior to receiving instructions for a medical intervention. In this manner, patient 4 may be empowered to take action, as needed, to address his or her medical status, which may help improve clinical outcomes for patient 4.

In the example illustrated by FIG. 5, computing system 20 includes a storage device 21, e.g., to store data retrieved from IMDs 10, and processing circuitry 23. Although not illustrated in FIG. 5 clinician computing devices 38 may similarly include a storage device and processing circuitry. Processing circuitry 23 may include one or more processors that are configured to implement functionality and/or process instructions for execution within computing systems 20. For example, processing circuitry 23 may be capable of processing instructions stored in storage device 21. Processing circuitry 23 may include, for example, microprocessors, DSPs, ASICs, FPGAs, or equivalent discrete or integrated logic circuitry, or a combination of any of the foregoing devices or circuitry. Accordingly, processing circuitry 23 may include any suitable structure, whether in hardware, software, firmware, or any combination thereof, to perform the functions ascribed herein to processing circuitry 98. Processing circuitry 23 of computing systems 20 and/or the processing circuitry of clinician computing devices 38 may implement any of the techniques described herein to analyze impedance values received from IMD 10, e.g., to determine a health condition status of patient 4 (e.g., worsening heart failure).

Storage device 21 may include a computer-readable storage medium or computer-readable storage device. In some examples, storage device 21 includes one or more of a short-term memory or a long-term memory. Storage device 21 may include, for example, RAM, DRAM, SRAM, magnetic discs, optical discs, flash memories, or forms of EPROM or EEPROM. In some examples, storage device 21 is used to store data indicative of instructions for execution by processing circuitry 23.

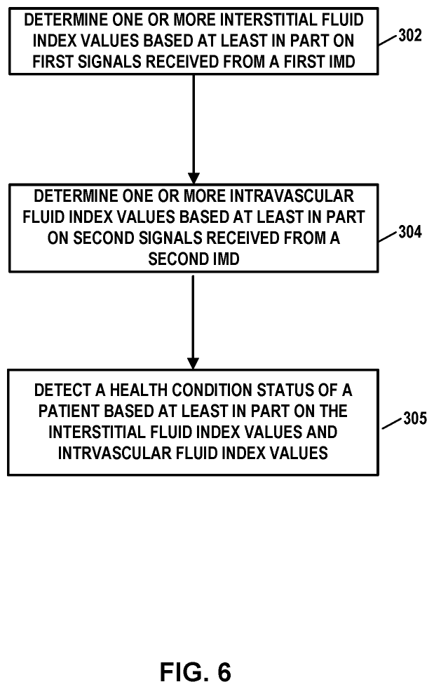

FIG. 6 is a flow diagram illustrating an example operation of detecting statuses of health conditions of a patient, in accordance with one or more techniques of this disclosure. The example operation of FIG. 6 may be performed by processing circuitry of any one of IMDs 10, computing device(s) 12, computing systems 20, clinician computing devices 38, or IoT devices 30 (e.g., by processing circuitry 50, 130, or 23), or by processing circuitry of two or more of these devices respectively performing portions of the example operation.

As seen in the example of FIG. 6, processing circuitry initially may determine one or more interstitial fluid index values based at least in part on one or more first signals received from a first IMD 10a (302). Processing circuitry may also determine one or more intravascular fluid index values based at least in part on one or more second signals received from a second IMD 10b (304). Processing circuitry may detect a health condition status of the patient based at least in part on the interstitial fluid index values and the intravascular fluid index values (305).

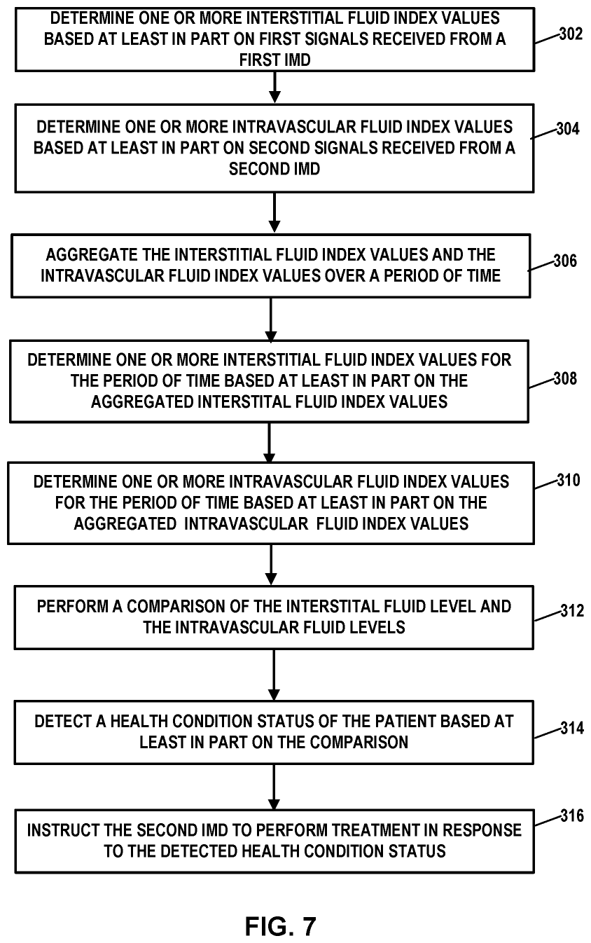

FIG. 7 is a flow diagram illustrating another example operation of detecting statuses of health conditions of a patient, in accordance with one or more techniques of this disclosure. The example operation of FIG. 7 may be performed by processing circuitry of any one of IMDs 10, computing device(s) 12, computing systems 20, clinician computing devices 38, or IoT devices 30 (e.g., by processing circuitry 50, 130, or 23), or by processing circuitry of two or more of these devices respectively performing portions of the example operation.

According to the example of FIG. 7, the processing circuitry may aggregate the interstitial fluid index values and the intravascular fluid index values (e.g., determined as described above with respect to items 302 and 304 of FIG. 6) over a period of time (306). Moreover, processing circuitry may determine one or more interstitial fluid levels for the period of time based at least in part on the aggregated interstitial fluid index values (308). Further, processing circuitry may determine one or more intravascular fluid levels for the period of time based at least in part on the aggregated fluid intravascular index values (310). Further, processing circuitry may perform a comparison of the interstitial fluid levels and the intravascular fluid levels (312). Moreover, processing circuitry may detect the health condition status of the patient based at least in part on the comparison (314). In some examples, processing circuitry may instruct the second IMD to perform treatment in response to the detected health condition status (316).

In some examples, processing circuitry may send the detected health condition status to one or more computing devices, such as clinician computing device 38, computing systems 20, computer device(s) 12, and IMDs 10. In some examples, processing circuitry may detect the health condition status based at least in part on the comparison and patient related information received from an external monitoring device. In some examples, processing circuitry may detect the health condition status based at least in part on the comparison and medical records received from a computing device. In some examples, results of the comparison may be inputs into a machine learning model to determine a health condition status such as a probability of HF and/or a probability of worsening HF. Such a model may also include one or more other inputs from any one or more devices described herein, such as, but not limited to, activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, respiratory effort, heart sounds, sleep apnea burden, PVC burden, QRST morphology changes, temperature, maximum volume of oxygen, blood glucose, peripheral perfusion, and sleep patterns.

The first signals may be received from one or more first sensors in the first IMD 10a and the second signals may be received from one or more second sensors in the second IMD 10b. The first signals may include a sensed subcutaneous impedance signal. The second signals may include a sensed intracardiac impedance signal and/or measured heart sounds, such as A1, A2, A3 and A4 (also referred to as S1, S2, S3 and S4), e.g., amplitude and slew measurements, that indicate intravascular fluid level of patient 4. The second sensors may include electrodes, and the one or more second signals may include an intracardiac impedance signal and/or measured heart sounds. The second IMD 10b may be an intracardiac pacemaker or a cardiac resynchronization therapy device. The first sensors may contact interstitial fluid.

In some examples, a machine learning model used to detect a health condition status, e.g., heart failure level or heart failure event risk, based on orthogonal impedance indices, and in some cases other patient parameters, may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model selection-techniques, such as Bayesian information criterion (BIC) or Akaike information criterion (AIC), may be used to evaluate probability models prior to use.

In some examples, an integrated diagnostics model may be used to determine a number of criteria that are met based on sensed or measured patient parameters, including intravascular and interstitial (subcutaneous) impedance. For example, the probability model may determine that X of Y criteria have been met with respect to the parameters. In such examples, Y may be the maximum number of criteria possible given the particular configuration of parameters the probability model is using, and X may be a variable less than or equal to Y that increments based on the parameters meeting certain criteria. In an illustrative example, the probability model may increment X in response to determining that the patient has a high heart rate indicating a high diagnostic state.

Processing circuitry may determine, from the respective parameter values, diagnostic states for each parameter. Processing circuitry may compare the heart rate score to one or more risk thresholds to determine a diagnostic state of high (H) risk, medium (M) risk, or low (L) risk, in some examples. In some examples, processing circuitry may determine a joint diagnostic state based on multiple parameters that are independent of one another.

In some examples, diagnostic states may include a finite number of potential diagnostic states for each parameter (e.g., very high, high, medium, low, very low, etc.). For example, the diagnostic states may include states of high risk, medium risk, or low risk, for each parameter. In some instances, one or more of the parameters can have a different number of potential diagnostic states (e.g., one state, two states, three states, or more), whereas other parameters may have a greater or lesser number of potential diagnostic states. For example, heart rate may have three diagnostic states (H, M, and L), whereas prior arrhythmia may have less than three diagnostic states (H and L). In other examples, diagnostic states may include a continuum or sliding spectrum of diagnostic state values, rather than discrete states. Diagnostic states of the parameters may be independent for each parameter. For example, a diagnostic state for a first set of one or more parameters may be independent of diagnostic states associated with one or more other parameters. In some examples, the probability mode framework, such as a BBN framework, may include additional parameters, where the respective values of the parameters are conditionally independent of one another.

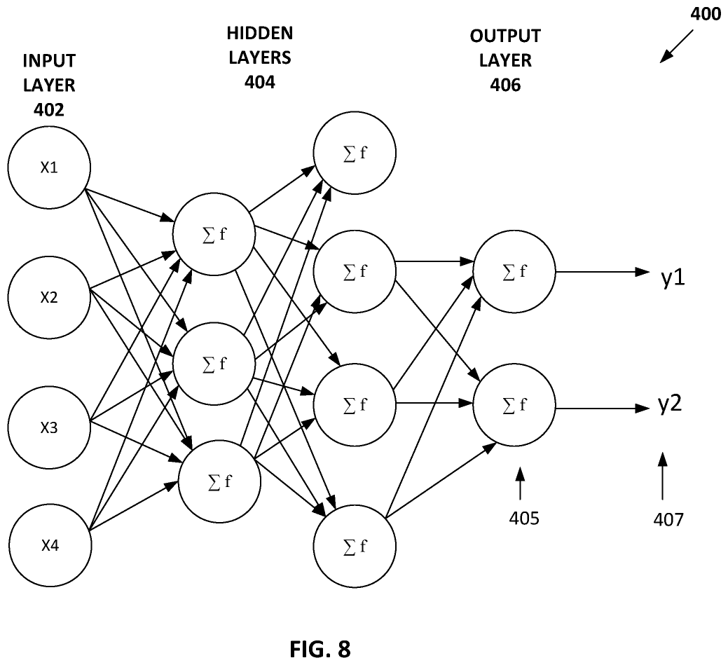

FIG. 8 is a conceptual diagram illustrating an example machine learning model 400 configured to determine one or more output values indicative of HF level or risk, or another health condition status, based on intravascular and interstitial fluid index values. Machine learning model 400 may correspond to rules 196 and 250. Machine learning model 400 is an example of a deep learning model, or deep learning algorithm, trained to determine whether a particular set of patient parameter data indicates a condition of the patient meriting a system response as described herein. One or more of IMD 10, computing device 12, or computing system 20 may train, store, and/or utilize machine learning model 400, but other devices may apply inputs associated with a particular patient to machine learning model 400 in other examples.

As shown in the example of FIG. 8, machine learning model 400 may include three layers. These three layers include input layer 402, hidden layer 404, and output layer 406. Output layer 406 comprises the output from the transfer function 405 of output layer 406. Input layer 402 represents each of the input values X1 through X4 provided to machine learning model 400. The number of inputs may be less than or greater than 4, including much greater than 4, e.g., hundreds or thousands. In some examples, the input values may any of the of values input into a machine learning model, as described above. In some examples, input values may include interstitial and intravascular fluid index values, or levels determination based on aggregation of these values. In addition, in some examples input values of machine learning model 400 may include additional data, such as data relating to one or more additional parameters of patient 4, e.g., values of parameters of heart sounds of patient 4.

Each of the input values for each node in the input layer 402 is provided to each node of hidden layer 404. In the example of FIG. 8, hidden layers 404 include two layers, one layer having four nodes and the other layer having three nodes, but fewer or greater number of nodes may be used in other examples. Each input from input layer 402 is multiplied by a weight and then summed at each node of hidden layers 404. During training of machine learning model 400, the weights for each input are adjusted to establish the relationship between the inputs determining whether a particular set of inputs represents a health event and/or determining a score indicative of whether a set of inputs may be representative of a health event. In some examples, one hidden layer may be incorporated into machine learning model 400, or three or more hidden layers may be incorporated into machine learning model 400, where each layer includes the same or different number of nodes.

The result of each node within hidden layers 404 is applied to the transfer function of output layer 406. The transfer function may be liner or non-linear, depending on the number of layers within machine learning model 400. Example non-linear transfer functions may be a sigmoid function or a rectifier function. The output 407 of the transfer function may be a classification that indicates whether the particular set of fluid index values or other inputs represents a health event and/or a score indicative of an extent to which the input data set represents a health event. By applying the fluid index data and/or other patient parameter data to a machine learning model, such as machine learning model 400, processing circuitry of system 2 is able to determine an HF or other health condition status of a patient with great accuracy, specificity, and sensitivity. This may facilitate alerts and other interventions as described herein.

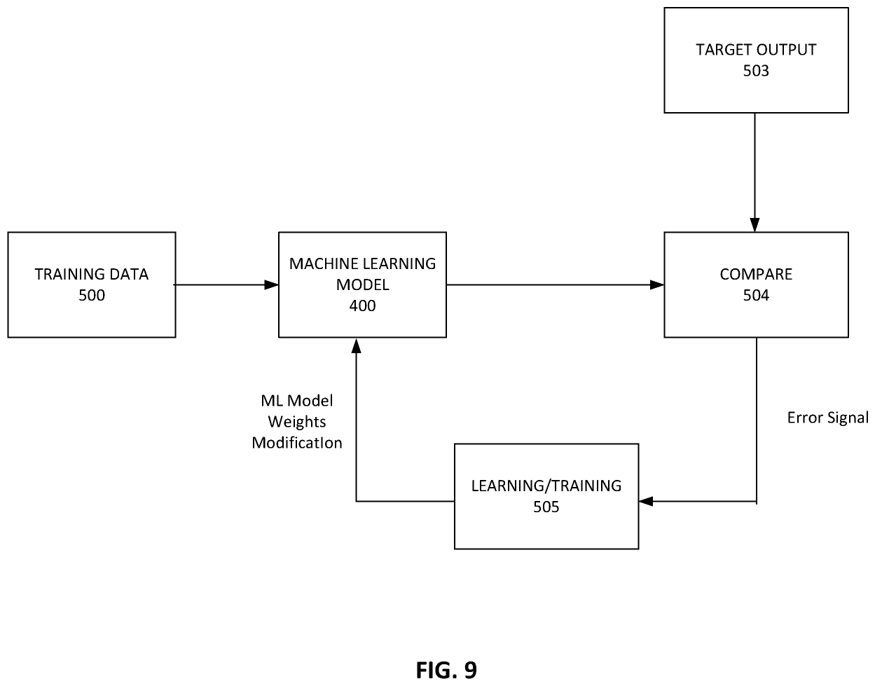

FIG. 9 is an example of a machine learning model 400 being trained using supervised and/or reinforcement learning techniques. Machine learning model 400 may be implemented using any number of models for supervised and/or reinforcement learning, such as but not limited to, an artificial neural network, a decision tree, naïve Bayes network, support vector machine, or k-nearest neighbor model, to name only a few of the examples discussed above. In some examples, processing circuitry one or more of IMD 10, computing device 12, and/or computing system 20 initially trains the machine learning model 400 based on training set data 500 including numerous instances of input data corresponding to different health conditions statuses, e.g., as labeled by an expert. A prediction or classification by the machine learning model 400 may be compared 504 to the target output 503, e.g., as determined based on the label. Based on an error signal representing the comparison, the processing circuitry implementing a learning/training function 505 may send or apply a modification to weights of machine learning model 400 or otherwise modify/update the machine learning model 400. For example, one or more of IMD 10, computing device 12, and/or computing system 20 may, for each training instance in the training set 500, modify machine learning model 400 to change a score generated by the machine learning model 400 in response to data applied to the machine learning model 400.

FIG. 9A is a perspective drawing illustrating an IMD 110A, which may be an example configuration of IMD 10A of FIG. 1 as an ICM. In the example shown in FIG. 9A, IMD 110A may be embodied as a monitoring device having housing 912, proximal electrode 56A and distal electrode 56B. Housing 912 may further comprise first major surface 914, second major surface 918, proximal end 920, and distal end 922. Housing 912 encloses electronic circuitry located inside the IMD 10A and protects the circuitry contained therein from body fluids. Housing 912 may be hermetically sealed and configured for subcutaneous implantation. Electrical feedthroughs provide electrical connection of electrodes 56A and 56B.

In the example shown in FIG. 9A, IMD 110A is defined by a length L, a width W and thickness or depth D and is in the form of an elongated rectangular prism wherein the length L is much larger than the width W, which in turn is larger than the depth D. In one example, the geometry of the IMD 110A-in particular a width W greater than the depth D-is selected to allow IMD 110A to be inserted under the skin of the patient using a minimally invasive procedure and to remain in the desired orientation during insertion. For example, the device shown in FIG. 9A includes radial asymmetries (notably, the rectangular shape) along the longitudinal axis that maintains the device in the proper orientation following insertion. For example, the spacing between proximal electrode 56A and distal electrode 56B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 110A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 914 may range from 3 mm to 15, mm, from 3 mm to 10 mm, or from 5 mm to 15 mm, and may be any single or range of widths between 3 mm and 15 mm. The thickness of depth D of IMD 110A may range from 2 mm to 15 mm, from 2 mm to 9 mm, from 2 mm to 5 mm, from 5 mm to 15 mm, and may be any single or range of depths between 2 mm and 15 mm. In addition, IMD 110A according to an example of the present disclosure is has a geometry and size designed for ease of implant and patient comfort. Examples of IMD 110A described in this disclosure may have a volume of three cubic centimeters (cm) or less, 1.5 cubic cm or less or any volume between three and 1.5 cubic centimeters.

In the example shown in FIG. 9A, once inserted within the patient, the first major surface 914 faces outward, toward the skin of the patient while the second major surface 918 is located opposite the first major surface 914. In addition, in the example shown in FIG. 9A, proximal end 920 and distal end 922 are rounded to reduce discomfort and irritation to surrounding tissue once inserted under the skin of the patient. IMD 110A, including instrument and method for inserting IMD 110A is described, for example, in U.S. Patent Publication No. 2014/0276928, incorporated herein by reference in its entirety.

Proximal electrode 56A is at or proximate to proximal end 920, and distal electrode 56B is at or proximate to distal end 922. Proximal electrode 56A and distal electrode 56B are used to sense cardiac EGM signals, e.g., ECG signals, and measure interstitial impedance thoracically outside the ribcage, which may be sub-muscularly or subcutaneously. EGM signals and impedance measurements may be stored in a memory of IMD 110A, and data may be transmitted via integrated antenna 26A to another device, which may be another implantable device or an external device, such as computing device 12. In some example, electrodes 56A and 56B may additionally or alternatively be used for sensing any bio-potential signal of interest, which may be, for example, an EGM, EEG, EMG, or a nerve signal, from any implanted location. Housing 912 may house the circuitry of IMD 10 illustrated in FIG. 2.

In the example shown in FIG. 9A, proximal electrode 56A is at or in close proximity to the proximal end 920 and distal electrode 56B is at or in close proximity to distal end 922. In this example, distal electrode 56B is not limited to a flattened, outward facing surface, but may extend from first major surface 914 around rounded edges 924 and/or end surface 926 and onto the second major surface 918 so that the electrode 56B has a three-dimensional curved configuration. In some examples, electrode 56B is an uninsulated portion of a metallic, e.g., titanium, part of housing 912.

In the example shown in FIG. 9A, proximal electrode 56A is located on first major surface 914 and is substantially flat, and outward facing. However, in other examples proximal electrode 56A may utilize the three dimensional curved configuration of distal electrode 56B, providing a three dimensional proximal electrode (not shown in this example). Similarly, in other examples distal electrode 56B may utilize a substantially flat, outward facing electrode located on first major surface 914 similar to that shown with respect to proximal electrode 56A.

The various electrode configurations allow for configurations in which proximal electrode 56A and distal electrode 56B are located on both first major surface 914 and second major surface 918. In other configurations, such as that shown in FIG. 9A, only one of proximal electrode 56A and distal electrode 56B is located on both major surfaces 914 and 918, and in still other configurations both proximal electrode 56A and distal electrode 56B are located on one of the first major surface 914 or the second major surface 918 (e.g., proximal electrode 56A located on first major surface 914 while distal electrode 56B is located on second major surface 918). In another example, IMD 110A may include electrodes on both major surface 914 and 918 at or near the proximal and distal ends of the device, such that a total of four electrodes are included on IMD 110A. Electrodes 56A and 56B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 9A, proximal end 920 includes a header assembly 928 that includes one or more of proximal electrode 56A, integrated antenna 26A, anti-migration projections 932, and/or suture hole 934. Integrated antenna 26A is located on the same major surface (i.e., first major surface 914) as proximal electrode 56A and is also included as part of header assembly 928. Integrated antenna 26A allows IMD 110A to transmit and/or receive data. In other examples, integrated antenna 26A may be formed on the opposite major surface as proximal electrode 56A, or may be incorporated within the housing 912 of IMD 110A. In the example shown in FIG. 9A, anti-migration projections 932 are located adjacent to integrated antenna 26A and protrude away from first major surface 914 to prevent longitudinal movement of the device. In the example shown in FIG. 9A, anti-migration projections 932 include a plurality (e.g., nine) small bumps or protrusions extending away from first major surface 914. As discussed above, in other examples anti-migration projections 932 may be located on the opposite major surface as proximal electrode 56A and/or integrated antenna 26A. In addition, in the example shown in FIG. 9A, header assembly 928 includes suture hole 934, which provides another means of securing IMD 110A to the patient to prevent movement following insertion. In the example shown, suture hole 934 is located adjacent to proximal electrode 56A. In one example, header assembly 928 is a molded header assembly made from a polymeric or plastic material, which may be integrated or separable from the main portion of IMD 110A.

FIG. 9B is a perspective drawing illustrating another IMD 110B, which may be another example configuration of IMD 10A from FIG. 1 as an ICM. IMD 110B of FIG. 9B may be configured substantially similarly to IMD 110A of FIG. 9A, with differences between them discussed herein.

IMD 110B may include a leadless, subcutaneously-implantable monitoring device, e.g. an ICM. IMD 110B includes housing having a base 940 and an insulative cover 942. Proximal electrode 56C and distal electrode 56D may be formed or placed on an outer surface of cover 942. Various circuitries and components of IMD 110B, e.g., described with respect to FIG. 2, may be formed or placed on an inner surface of cover 942, or within base 940. In some examples, a battery or other power source of IMD 110B may be included within base 940. In the illustrated example, antenna 26B is formed or placed on the outer surface of cover 942, but may be formed or placed on the inner surface in some examples. In some examples, insulative cover 942 may be positioned over an open base 940 such that base 940 and cover 942 enclose the circuitries and other components and protect them from fluids such as body fluids. The housing including base 940 and insulative cover 942 may be hermetically sealed and configured for subcutaneous implantation.

Circuitries and components may be formed on the inner side of insulative cover 942, such as by using flip-chip technology. Insulative cover 942 may be flipped onto a base 940. When flipped and placed onto base 940, the components of IMD 110B formed on the inner side of insulative cover 942 may be positioned in a gap 944 defined by base 940. Electrodes 56C and 56D and antenna 26B may be electrically connected to circuitry formed on the inner side of insulative cover 942 through one or more vias (not shown) formed through insulative cover 942. Insulative cover 942 may be formed of sapphire (i.e., corundum), glass, parylene, and/or any other suitable insulating material. Base 940 may be formed from titanium or any other suitable material (e.g., a biocompatible material). Electrodes 56C and 56D may be formed from any of stainless steel, titanium, platinum, iridium, or alloys thereof. In addition, electrodes 56C and 56D may be coated with a material such as titanium nitride or fractal titanium nitride, although other suitable materials and coatings for such electrodes may be used.

In the example shown in FIG. 9B, the housing of IMD 110B defines a length L, a width W and thickness or depth D and is in the form of an elongated rectangular prism wherein the length L is much larger than the width W, which in turn is larger than the depth D, similar to IMD 110A of FIG. 9A. For example, the spacing between proximal electrode 56C and distal electrode 56D may range from 5 mm to 50 mm, from 30 mm to 50 mm, from 35 mm to 45 mm, and may be any single spacing or range of spacings from 5 mm to 50 mm, such as approximately 40 mm. In addition, IMD 110B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 110B may range from 2 mm to 15 mm, from 5 mm to 15 mm, or from 3 mm to 5 mm, and may be any single depth or range of depths between 2 mm and 15 mm, such as approximately 4 mm. IMD 110B may have a volume of three cubic centimeters (cm) or less, or 1.5 cubic cm or less, such as approximately 1.4 cubic cm.

In the example shown in FIG. 9B, once inserted subcutaneously within the patient, outer surface of cover 942 faces outward, toward the skin of the patient. In addition, as shown in FIG. 9B, proximal end 946 and distal end 948 are rounded to reduce discomfort and irritation to surrounding tissue once inserted under the skin of the patient. In addition, edges of IMD 110B may be rounded.

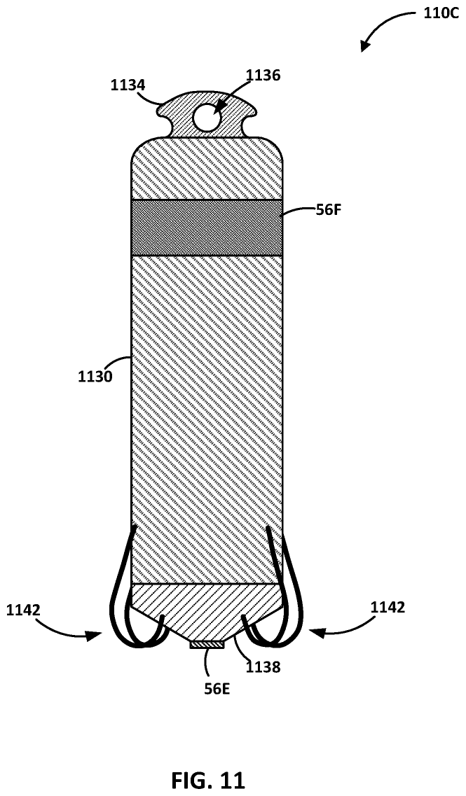

FIG. 11 is a conceptual drawing illustrating an example configuration an IMD 110C, which may be an example of IMD 10B of FIG. 1 configured as an intracardiac pacing device. As shown in FIG. 11, IMD 110C includes case 1130, cap 1138, electrode 56E, electrode 56F, fixation mechanisms 1142, flange 1134, and opening 1136. Together, case 1130 and cap 1138 may be considered the housing of IMD 110C. In this manner, case 1130 and cap 1138 may enclose and protect the various electrical components, e.g., circuitry, within IMD 110C. Case 1130 may enclose substantially all of the electrical components, and cap 1138 may seal case 1130 and create the hermetically sealed housing of IMD 110C. Although IMD 110C is generally described as including one or more electrodes, IMD 110C may typically include at least two electrodes (e.g., electrodes 56E and 56F) to deliver an electrical signal (e.g., therapy such as cardiac pacing) and/or provide at least one sensing vector.

Electrodes 56E and 56F are carried on the housing created by case 1130 and cap 1138. In this manner, electrodes 56E and 56F may be considered leadless electrodes. In the example of FIG. 11, electrode 56E is disposed on the exterior surface of cap 1138. Electrode 56E may be a circular electrode positioned to contact cardiac tissue upon implantation. Electrode 56F may be a ring or cylindrical electrode disposed on the exterior surface of case 1130. Both case 1130 and cap 1138 may be electrically insulating.

Electrode 56E may be used as a cathode and electrode 56F may be used as an anode, or vice versa, for delivering cardiac pacing. However, electrodes 56E and 56F may be used in any stimulation configuration. In addition, electrodes 56E and 56F may be used to detect intrinsic electrical signals from cardiac muscle, and to measure intravascular, e.g., intracardiac, impedance.

Fixation mechanisms 1142 may attach IMD 110C to cardiac tissue. Fixation mechanisms 1142 may be active fixation tines, screws, clamps, adhesive members, or any other mechanisms for attaching a device to tissue. As shown in the example of FIG. 11, fixation mechanisms 1142 may be constructed of a memory material, such as a shape memory alloy (e.g., nickel titanium), that retains a preformed shape. During implantation, fixation mechanisms 1142 may be flexed forward to pierce tissue and allowed to flex back towards case 1130. In this manner, fixation mechanisms 1142 may be embedded within the target tissue.

Flange 1134 may be provided on one end of case 1130 to enable tethering or extraction of IMD 110C. For example, a suture or other device may be inserted around flange 1134 and/or through opening 1136 and attached to tissue. In this manner, flange 1134 may provide a secondary attachment structure to tether or retain IMD 110C within the heart if fixation mechanisms 1142 fail. Flange 1134 and/or opening 1136 may also be used to extract IMD 110C once the IMD needs to be explanted (or removed) from a patient.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes a first implantable medical device (IMD) comprising one or more first sensors, the first IMD configured to receive one or more first signals from the one or more first sensors to determine one or more interstitial fluid index values based at least in part on the received first signals; and a second IMD comprising one or more second sensors, the second IMD configured to receive one or more second signals from the second sensors to determine one or more intravascular fluid index values based at least in part on the received second signals; and processing circuitry configured to detect a health condition status of a patient based at least in part on the interstitial fluid index values and the intravascular fluid index values.

Example 2: The system of example 1, wherein the first IMD is configured to be inserted subcutaneously, the first sensors include electrodes, and the first signals include a sensed subcutaneous impedance.

Example 3: The system of any of examples 1 and 2, wherein the second sensors include electrodes, and the second signals include one or more of a sensed intracardiac impedance and sensed heart sounds.

Example 4: The system of example 3, wherein the second IMD comprises an intracardiac pacemaker or a cardiac resynchronization therapy device.

Example 5: The system of any of examples 2 through 4, wherein at least one of the electrodes of the first IMD contacts interstitial fluid.

Example 6: The system of any of examples 1 through 5, wherein the second IMD is configured to be inserted into a heart of the patient, and the second signals include one or more of a sensed intracardiac impedance and second heart sounds.

Example 7: The system of any of examples 2 through 6, wherein the second IMD is configured to be inserted into a heart of the patient, and the second signals include one or more of a sensed intracardiac impedance and second heart sounds.

Example 8: The system of any of examples 2 through 7, wherein the processing circuitry is configured to detect a heart failure level based on the interstitial fluid index values and the intravascular fluid index values.

Example 9: The system of any of examples 7 and 8, wherein the processing circuitry is configured to detect a heart failure level based on the interstitial fluid index values and the intravascular fluid index values.

Example 10: The system of any of examples 1 through 9, wherein the processing circuitry is further configured to: aggregate the interstitial fluid index values and the intravascular fluid index values over a period of time; determine one or more interstitial fluid levels for the period of time based at least in part on the aggregated interstitial fluid index values; determine one or more intravascular fluid levels for the period of time based at least in part on the aggregated fluid intravascular index values; and perform a comparison of the interstitial fluid levels and the intravascular fluid levels; and detect the health condition status of the patient based at least in part on the comparison.

Example 11: The system of example 10, the system further comprising an external monitoring device to monitor and obtain patient related information, wherein processing circuitry is further configured to detect the health condition status based at least in part on the comparison and the obtained patient related information.

Example 12: The system of any of examples 10 and 11, wherein processing circuitry is further configured to receive medical records of the patient and detect the health condition status based at least in part on the comparison and on the received medical records.

Example 13: The system of any of examples 1 through 12, wherein the processing circuitry is located in a computing system external from the patient and the processing circuitry determines the one or more interstitial fluid index values and the one or more intravascular fluid index values.

Example 14: The system of example 13, wherein operations of the processing circuitry are configured to be operated through an application of a smartphone.

Example 15: The system of any of examples 1 through 14, wherein the second IMD is configured to perform treatment in response to the detected health condition status.

Example 16: A method of detecting statuses of health conditions of a patient includes determining one or more interstitial fluid index values based at least in part on one or more first signals received from a first implantable medical device (IMD); determining one or more intravascular fluid index values based at least in part on one or more second signals received from a second IMD; and detecting a health condition status of the patient based at least in part on the interstitial fluid index values and the intravascular fluid index values.

Example 17: The method of example 16, wherein the first signals are received from one or more first sensors in the first IMD and the second signals are received from one or more second sensors in the second IMD.

Example 18: The method of any of examples 16 and 17, wherein the first signals include a sensed subcutaneous impedance signal.

Example 19: The method of example 18, wherein the first IMD is inserted subcutaneously in the patient.

Example 20: The method of any of examples 16 through 19, wherein the second signals include a sensed intracardiac impedance signal and a sensed heart sounds signal.

Example 21: The method of example 20, wherein the second IMD is inserted into a heart of the patient.

Example 22: The method of any of examples 19 through 21, wherein the second IMD is inserted into a heart of the patient and the second signals include a sensed intracardiac impedance signal and a sensed heart sounds signal.

Example 23: The method of any of examples 16 through 22, wherein the second sensors include electrodes, and the second signals include one or more of an intracardiac impedance signal.

Example 24: The method of any of examples 20 through 23, wherein the second IMD is an intracardiac pacemaker or a cardiac resynchronization therapy device.

Example 25: The method of any of examples 17 through 24, wherein at least one of the first sensors contacts interstitial fluid.

Example 26: The method of any of examples 18 through 25, further comprising detecting a heart failure level based on the interstitial fluid index values and the intravascular fluid index values.

Example 27: The method of any of examples 21 through 26, further comprising detecting a heart failure level based on the interstitial fluid index values and the intravascular fluid index values.

Example 28: The method of any of examples 22 through 27, further comprising detecting a heart failure level based on the interstitial fluid index values and the intravascular fluid index values.

Example 29: The method of any of examples 16 through 28, further includes aggregating the interstitial fluid index values and the intravascular fluid index values over a period of time; determining one or more interstitial fluid levels for the period of time based at least in part on the aggregated interstitial fluid index values; determining one or more intravascular fluid levels for the period of time based at least in part on the aggregated fluid intravascular index values; and performing a comparison of the interstitial fluid levels and the intravascular fluid levels; and detecting the health condition status of the patient based at least in part on the comparison.

Example 30: The method of example 29, further includes detecting the health condition status based at least in part on the comparison and patient related information received from an external monitoring device.

Example 31: The method of any of examples 29 and 30, further includes detecting the health condition status based at least in part on the comparison and medical records received from a computing device.

Example 32: The method of any of examples 29 through 31, further includes instructing the second IMD to perform treatment in response to the detected health condition status.

Example 33: The method of any of examples 16 through 32, wherein the method is executed by processing circuitry.

Example 34: A non-transitory computer-readable storage medium includes determine one or more interstitial fluid index values based at least in part on one or more first signals received from a first implantable medical device (IMD); determine one or more intravascular fluid index values based at least in part on one or more second signals received from a second IMD; and detect a health condition status of the patient based at least in part on the interstitial fluid index values and the intravascular fluid index values, wherein the first signals include a subcutaneous impedance signal and the second signals include one or more of an intracardiac impedance signal and a heart sounds signal.

Various examples have been described. These and other examples are within the scope of the following claims.

1. A system comprising:
a first implantable medical device (IMD) comprising one or more first sensors, the first IMD configured to receive one or more first signals from the one or more first sensors to determine one or more interstitial fluid index values based at least in part on the received first signals; and
a second IMD comprising one or more second sensors, the second IMD configured to receive one or more second signals from the second sensors to determine one or more intravascular fluid index values based at least in part on the received second signals; and
processing circuitry configured to detect a health condition status of a patient based at least in part on the interstitial fluid index values and the intravascular fluid index values.

2. The system of claim 1, wherein the first IMD is configured to be inserted subcutaneously, the one or more first sensors include electrodes, and the one or more first signals include a sensed subcutaneous impedance.

3. The system of claim 2, wherein at least one of the electrodes of the first IMD contacts interstitial fluid.

4. The system of claim 2, wherein the one or more second sensors include electrodes, and the one or more second signals include one or more of a sensed intracardiac impedance or sensed heart sounds.

5. The system of claim 4, wherein the second IMD comprises an intracardiac pacemaker.

6. The system of claim 1, wherein the health condition status comprises at least one of a heart failure level or a risk level of a heart failure event.

7. The system of claim 1, wherein to detect the health condition status of the patient based at least in part on the interstitial fluid index values and the intravascular fluid index values, the processing circuitry is configured to:
aggregate the interstitial fluid index values and the intravascular fluid index values over a period of time;
determine one or more interstitial fluid levels for the period of time based at least in part on the aggregated interstitial fluid index values;
determine one or more intravascular fluid levels for the period of time based at least in part on the aggregated intravascular fluid index values;
compare the interstitial fluid levels and the intravascular fluid levels; and
detect the health condition status of the patient based at least in part on the comparison.

8. The system of claim 7, further comprising an external monitoring device configured to sense one or more physiological signals and determine patient related information based on the physiological signals, wherein processing circuitry is further configured to detect the health condition status based at least in part on the comparison and the patient related information.

9. The system of claim 7, wherein processing circuitry is further configured to receive medical records of the patient and detect the health condition status based at least in part on the comparison and on the received medical records.

10. The system of claim 1, wherein the processing circuitry is located in a computing system external from the patient.

11. The system of claim 10, wherein the processing circuitry determines the one or more interstitial fluid index values and the one or more intravascular fluid index values.

12. The system of claim 10, wherein the computing system comprises at least one of a smartphone or a cloud computing system.

13. The system of claim 1, wherein the processing circuitry is located within one of the first IMD or the second IMD.

14. The system of claim 1, wherein the second IMD is configured to perform treatment of the patient in response to the detected health condition status.

15. The system of claim 1, wherein the processing circuitry is configured to detect the health condition status of the patient based on application of the interstitial fluid index values and the intravascular fluid index values to a machine learning model.

16. The system of claim 15, wherein the processing circuitry is configured to detect the health condition status of the patient based on application of values of one or more additional patient parameters to the machine learning model.

17. The system of claim 16, wherein the one or more additional patient parameters comprises heart sounds sensed by the second IMD.

18. The system of claim 1, wherein the first IMD comprises an insertable cardiac monitor comprising a housing configured for subcutaneous implantation in a patient, the housing having a length between 40 millimeters (mm) and 60 mm between a first end and a second end, a width less than the length, and a depth less than the width,
wherein the one or more first sensors comprise a first electrode at or proximate to the first end and a second electrode at or proximate to the second end.

19. A method of detecting statuses of health conditions of a patient, the method comprising:
determining one or more interstitial fluid index values based at least in part on one or more first signals received from a first implantable medical device (IMD);
determining one or more intravascular fluid index values based at least in part on one or more second signals received from a second IMD; and
detecting a health condition status of the patient based at least in part on the interstitial fluid index values and the intravascular fluid index values.

20. The method of claim 19, further comprising:
aggregating the interstitial fluid index values and the intravascular fluid index values over a period of time;
determining one or more interstitial fluid levels for the period of time based at least in part on the aggregated interstitial fluid index values;
determining one or more intravascular fluid levels for the period of time based at least in part on the aggregated fluid intravascular index values;
performing a comparison of the interstitial fluid levels and the intravascular fluid levels; and
detecting the health condition status of the patient based at least in part on the comparison.
